(19) United States
(12) Patent Application Publication
(10) Pub. No.: US 2014/0057800 A1
(43) Pub. Date: Feb. 27, 2014

(54) SINGLE NUCLEOTIDE POLYMORPHISM ASSOCIATED WITH RISK OF INSULIN RESISTANCE DEVELOPMENT

(71) Applicants: Hans-Richard Brattbakk, Radal (NO); Ingerid Arbo, Trondheim (NO); Berit Johansen, Trondheim (NO); Mette Langaas, Trondheim (NO)

(72) Inventors: Hans-Richard Brattbakk, Radal (NO); Ingerid Arbo, Trondheim (NO); Berit Johansen, Trondheim (NO); Mette Langaas, Trondheim (NO)

(73) Assignee: Norwegian Univeristy of Science and Technology (NTNU), Trondheim (NO)

(21) Appl. No.: 13/994,596

(22) PCT Filed: Dec. 13, 2011
(86) PCT No.: PCT/IB11/03038

§ 371 Date: Oct. 16, 2013

Related U.S. Application Data
(60) Provisional application No. 61/424,005, filed on Dec. 16, 2010.

(51) Int. Cl. C12Q 1/68

(52) U.S. Cl. CPC C12Q 1/6883
USPC 506/9

(57) ABSTRACT

The present invention is directed to methods of identifying quantitative trait loci (QTL) markers associated with insulin resistance, and use of these markers to explain individual physiological responses to dietary glycemic load. In addition, expressional QTLs (eQTLs) have been identified to characterize the contribution of the genotype to variations in gene expression.

The present invention pertains to different genetic markers of importance to the molecular mechanism involved in insulin resistance. A number of SNPs (single nucleotide polymorphisms) that are associated with insulin resistance have been located in the gene vesicle associated membrane protein-associated protein A (VAPA). Individual responses to a dietary challenge are expected to vary among individuals. Individuals with either a weak or strong response in insulin resistance upon dietary changes in glycemic load showed distinct genotype profiles. These markers have been extensively screened and connections between variation in SNPs and changes in insulin resistance in response to diets with different glycemic load have been identified.

An association between genetic variability in VAPA and insulin resistance has been found where several specific SNPs on identified quantitative trait loci (QTLs) are pinpointed.

Susceptibility loci traits for insulin resistance and SNPs which are involved in the molecular mechanism of the VAPA genetic interactions with insulin resistance have been identified. The protein encoded by this gene is a type IV membrane protein. It is present in the plasma membrane and intracellular vesicles. It may also be associated with the cytoskeleton. This protein may function in vesicle trafficking, membrane fusion, protein complex assembly and cell motility. Alternative splicing occurs at this locus and two transcript variants encoding distinct isoforms have been identified.

One aspect of the present invention is directed to specific SNPs as new markers of candidate QTLs related to genetic aspects of developing insulin resistance. Another aspect of the present invention involves the use of VAPA and plasma protein inhibitor of activated STAT-1 (PIAS1) as candidate genes for molecular mechanisms involved in insulin resistance. Yet another aspect of the present invention involves a specific marker SNP in the GIP (gastric inhibitory polypeptide) gene, a candidate expressional QTL (eQTL) affecting plasma plasminogen activator inhibitor-1 (PAI-1) concentrations related to insulin resistance.

The identified genetic markers can be used in the diagnosis of insulin resistance correlated with dietary diseases, especially glycemic loads. Furthermore such markers can be used in developing suitable drugs for regulating glycemic response in people with such diseases.

Furthermore, such markers associated with insulin resistance can be used to explain individual physiological responses to dietary glycemic load. SNP typing can be used to provide concrete dietary advice to persons genetically predisposed to type II diabetes (T2D).

### BACKGROUND

Type 2 diabetes (T2D) is defined as chronic hyperglycemia, manifested when insulin production is overwhelmed by insulin resistance in target cells, leading to a decreased ability of glucose uptake (Tripathy and Chavez, Curr Diab Rep, 2010, 10(3): pp. 184-91, incorporated herein by reference). Insulin resistance, however, precedes the onset of T2D by many years (Pagel-Langenickel et al., Endocr Rev, 2010, 31(1): pp. 25-51, incorporated herein by reference), and in addition to be a risk factor for T2D it is also an independent predictor for e.g. hypertension, coronary heart disease (CHD), stroke, and cancer (Facchini et al., J Clin Endocrinol Metab, 2001, 86(8): pp. 3574-8, incorporated herein by reference). Even though obesity is associated with increased insulin resistance, individuals of normal weight do also experience variable sensitivity to insulin (McLaughlin et al., Metabolism, 2004, 53(4): pp. 495-9, incorporated herein by reference).

Already 30 years ago it was stated that the prevalence of T2D could be reduced by lifestyle changes, but so far the incidence of T2D has only been increasing, and the expansion is now called a modern epidemic (Meigs, Diabetes Care, 2010, 33(8): pp. 1865-71, incorporated herein by reference). There are at least two plausible explanations for this: Firstly, the dietary guidelines may be underestimating the influence of dietary glycemic load on hyperinsulinemia (Ludwig, Jama, 2002, 287(18): pp. 2414-23, incorporated herein by reference). Secondly, the same guidelines may be too general. The capability to study the complex genetics behind interindividual metabolic differences (Lairon et al., Public Health Nutr, 2009, 12(9A): pp. 1601-6, incorporated herein by reference) has been developed only recently, revealing benefits of personalized nutrition among high-risk persons (Kaput, J., Curr Opin Biotechnol, 2008, 19(2): pp. 110-20; Martinez et al., Asia Pac J Clin Nutr, 2008, 17 Suppl 1: p. 119-22; both incorporated herein by reference).

Insulin resistance is a pathophysiological trait characterised by an aberrant blood lipid profile, endothelial dysfunction, increased plasma concentration of procoagulant factors, and markers of inflammation (Goldberg, R. B., J Clin Endocrinol Metab, 2009, 94(9): pp. 3171-82, incorporated herein by reference). The etiology of insulin resistance is complex and unlikely to be the same in every individual. A major determinant, though, seems to be cytokine induced activation of proinflammatory pathways in insulin target cells, reducing insulin sensitivity. This activates and attracts immune cells, and establishes a feed forward loop resulting in macrophage infiltration of the tissue, and additional cytokine secretion (Olefsky and Glass, Annu Rev Physiol, 2010, 72: pp. 219-46, incorporated herein by reference). The inflammatory origin can be retraced to cellular stress, caused by metabolic imbalance, hence, called metaflammation (Hotamisligil, Nature, 2006, 444(7121): pp. 860-7, incorporated herein by reference). Prolonged malnutrition leads to chronic metaflammation, and may eventually cause degeneration of tissue, and onset of disease (Kushner et al., Arthritis Care Res (Hoboken), 2010, 62(4): pp. 442-6, incorporated herein by reference). Hyperglycaemia and hyperinsulinemia following a meal rich in easily digested carbohydrates are associated with cellular stress and increase of inflammatory markers (O'Keefe et al., J Am Coll Cardiol, 2008, 51(3): pp. 249-55, incorporated herein by reference). Diets with low glycemic load and glycemic index are suggested to silence metaflammation, and subsequently increase insulin sensitivity (Barclay et al., Am J Clin Nutr, 2008, 87(3): pp. 627-37; McKeown et al., Diabetes Care, 2004, 27(2): pp. 538-46; and Qi and Hu, Curr Opin Lipidol, 2007, 18(1): pp. 3-8; all incorporated herein by reference).

Current evidence suggests that insulin resistance and the associated abnormalities constitute complex phenotypes, explained by both environmental and genetic factors. The genetic makeup underlying these traits consists of several quantitative trait loci (QTL), whereof each QTL only explains a small fraction of the phenotype. The limited effects of these individual QTL make them difficult to identify, but the list of allelic variants associated with susceptibility to T2D development, in terms of single nucleotide polymorphisms (SNPs), is growing (Voight, B. F., et al., Twelve type 2 diabetes susceptibility loci identified through large-scale association analysis. Nat Genet, 2010. 42(7): p. 579-89, incorporated herein by reference). Also SNPs associated directly with insulin resistance have been found, but this line of research is in an early phase. (See Kantartzis et al., Clin Sci (Lond), 2009, 116(6): pp. 531-7; Liu et al., J Clin Endocrinol Metab, 2009, 94(9): pp. 3575-82; Palmer et al., Diabetes, 2004, 53(11): pp. 3013-9; Richardson et al., Diabetologia, 2006, 49(10): pp. 2317-28; Ruchat et al., Diabet Med, 2008, 25(4): pp. 400-6; and Smith et al., Diabetes, 2003, 52(7): pp. 1611-8; all incorporated herein by reference.)

The expression of a gene is the most basic phenotype in an organism. The genotype determines complex phenotypic traits through expression of several genes: expressional QTL (eQTL) (Jansen and Nap, Trends Genet, 2001, 17(7): pp. 388-91; and Schadt et al., Nature, 2003, 422(6929): pp. 297-302, both incorporated herein by reference). eQTL provide a direct link between genotype variation and gene- or pathway activities. The motivation to study how SNPs associated with a disease or a phenotypic trait may affect gene expression is to gain a direct understanding of the molecular mechanisms affected by the allelic variation (Rockman and Kruglyak, Nat Rev Genet, 2006, 7(11): pp. 862-72, incorporated herein by reference).

Homeostatic model assessment (HOMA) is a method for assessing surrogate measures of pancreatic β-cell function, insulin sensitivity, and insulin resistance derived from fasting blood glucose and insulin, alternatively insulin connecting peptide (C-peptide) concentrations (Wallace et al., Diabetes Care, 2004, 27(6): pp. 1487-95, incorporated herein by reference). The model was first proposed in 1985 (Matthews, et al., Diabetologia, 1985, 28(7): pp. 412-9, incorporated herein by reference), and an updated computer model (HOMA2) was published in 1998 (Levy et al., Diabetes Care, 1998, 21(12): pp. 2191-2, incorporated herein by reference). The calculation of insulin resistance designated as HOMA2 IR, is calibrated to a reference population, where the value 1 is set as normal (Wallace et al., 2004). HOMA2 IR was found to be a significant determinant of insulin resistance (Mojiminiyi et al., Clin Chem Lab Med, 2010, incorporated herein by reference).

In the present study, performed on modestly overweight but otherwise healthy individuals, associations between variation in SNPs and changes in insulin resistance in response to diets with different glycemic load were examined. SNPs were linked to genes and biological functions to develop an understanding of the molecular mechanisms potentially involved in onset of insulin resistance.

### METHODS

### Subjects and Study Outline

A randomized, controlled cross-over diet intervention trial was conducted on thirty-two young and healthy women and men, with body mass index (BMI, in kg/m²) between 24.5 and 27.5. Iso- and normocaloric meal replacement diets (MRDs) constituted all nutrients consumed during the study periods of two times six days with an eight day wash-out period in-between. Fasting blood samples were collected before and after each diet period, and effects of dietary intake on leukocyte gene expression profiles and insulin resistance were analyzed, as described previously ((Arbo I, Brattbakk H R, Langaas M et al. A balanced macronutrient diet induces changes in a host of pro-inflammatory biomarkers, rendering a more healthy phenotype; a randomized cross-over trial, 2010, (manuscript submitted), incorporated herein by reference). The two MRDs were: a high-carbohydrate diet (AHC) composed of 65:15:20 energy percent (E %) of carbohydrates, proteins, and fats; and a moderate-carbohydrate diet (BMC) with 27:30:43 E % of carbohydrates, proteins, and fats. The glycemic load of the AHC diet was calculated to be 2.71 times higher than the BMC diet.

Data extracted from samples were grouped and coded, according to diet and time of sampling. The abbreviations AHC0, AHC6, BMC0, and BMC6 denote before (day 0) and after (day 6) the AHC and the BMC diet intervention, respectively. Pair-wise analyses of data were performed for four different comparisons, which will be referred to throughout this paper: 1) AHC6-AHC0 and 2) BMC6-BMC0 identified responses to the AHC and the BMC diets, respectively, during six days on the respective diets. The comparison 3) BMC6-AHC6 identified the differences between the end-point responses to diet AHC and BMC after six days on diet, and finally, 4) (BMC6-BMC0)-(AHC6-AHC0) identified differences between the responses to AHC and BMC dieting. Complementary and more detailed information about subject recruitment, exclusion criteria, subject baseline characteristics, MRD compositions, and sampling techniques were described previously (Arbo I, Brattbakk H R, Langaas M et al. A balanced macronutrient diet induces changes in a host of pro-inflammatory biomarkers, rendering a more healthy phenotype; a randomized cross-over trial, 2010, (manuscript submitted), incorporated herein by reference).

### Microarray Hybridization and Data Analysis

Microarray analysis and preprocessing of microarray data was performed as previously described (Arbo I, Brattbakk H R, Langaas M et al. A balanced macronutrient diet induces changes in a host of pro-inflammatory biomarkers, rendering a more healthy phenotype; a randomized cross-over trial, 2010, (manuscript submitted), incorporated herein by reference). Briefly, leukocyte gene expression profiling was done on the HumanHT-12 Expression BeadChip v3.0 (Illumina). After removal of two outlier samples, background correction based on negative controls, quantile-quantile normalization, signal log₂-transformation, and removal of not detected or bad probes, 27 372 unique probes were left in the “gene expression dataset”. The paired analyses of AHC6-AHC0 and BMC6-BMC0 identified 3225 and 1370 differentially expressed genes, respectively, where 843 genes overlapped between the analyses. For the paired groups BMC6-AHC6 and (BMC6-BMC0)-(AHC6-AHC0), no differentially expressed genes were identified. Microarray data were submitted to ArrayExpress (www.ebi.ac.uk/arrayexpress, accession number: E-TABM-1073).

### Analysis of the Bio-Plex Diabetes Panel and Assessment of Insulin Resistance

Protein concentration analyses and assessment of insulin resistance were performed as previously described (Arbo I, Brattbakk H R, Langaas M et al. A balanced macronutrient diet induces changes in a host of pro-inflammatory biomarkers, rendering a more healthy phenotype; a randomized cross-over trial, 2010, (manuscript submitted), incorporated herein by reference), using fasting EDTA-plasma samples. Bio-Plex Diabetes Panel assays (Bio-Rad Laboratories Inc., Hercules, Calif., USA) were performed using Luminex xMAP™ technology, with a Bio-Plex 200 suspension array reader, and the data was extracted with the Bio-Plex Manager 5.0 software (Bio-Rad Laboratories Inc.). Briefly, analysis of the paired groups showed decreased (P<0.05) plasma concentrations of visfatin (nicotinamide phosphoribosyltransferase, Nampt), and increased plasma concentration of resistin (P<0.05) during AHC dieting. Likewise, during BMC dieting the analysis showed decreased plasma concentrations of insulin, C-peptide, glucagon, plasminogen activator inhibitor-1 (PAI-1), glucagon-like peptide-1 (GLP-1), tumor necrosis factor-α (TNF), interleukin-6 (IL-6), and visfatin, and increased plasma concentrations of resistin. Gastric inhibitory polypeptide (GIP), ghrelin, and leptin did not respond to any of the diet interventions.

The HOMA2 calculator version 2.2.2® (Diabetes Trials Units, University of Oxford, www.dtu.ox.ac.uk/homacalculator/index.php) (Matthews et al., 1985) was used to determine changes in insulin resistance in terms of HOMA2 IR. There was an average decrease in HOMA2 IR during both the AHC diet and the BMC diet, but the downregulation was only significant during BMC dieting.

### Genotyping

DNA was extracted from EDTA-blood using E.Z.N.A Blood DNA Kit (D3392, OMEGA Bio-Tek, Inc., Norcross, Ga., USA). The subjects were genotyped using the ˜200 K Cardio-MetaboChip (Metabochip) SNP array, an Infinium iSelect HD Custom Genotyping BeadChip (Illumina, San Diego, Calif., USA), designed by the Cardio-MetaboChip Consortium (Broad Institute, Cambridge, Mass., USA), and analyzed according to the Infinium HD Assay Ultra, Manual Experienced User Card. The Metabochip consists of SNPs associated with diseases or traits relevant to metabolic and atherosclerosis-cardiovascular endpoints, including T2D and hyperglycemia. The BeadChips were read by a BeadArray™ reader, and data were exported to GenomeStudio™ V2009, Genotyping V1.1.9 (Illumina), for visual quality control of genotype clustering, and extraction of quality measures (ChiTest100 and GenTrain Score) (Illumina, GenomeStudio™ Genotyping Module v1.0 User Guide. 2008, Illumina, Inc: San Diego, Calif., incorporated herein by reference). The ChiTest100 is a p-value calculated for each SNP, reflecting the deviation of that SNP to the genotype distribution according to the Hardy-Weinberg Equilibrium (HWE), using the χ² statistic, normalized to 100 subjects. GenTrain Score is a measure of SNP clustering performance indicated by a number increasing with cluster quality, form 0 to 1.

### Candidate Gene Selection

A set of 22 transcription regulators and seven ligand-dependent nuclear receptors central to insulin resistance development (Olefsky and Glass, 2010; Hotamisligil, 2006; and Wymann and Schneiter, Nat Rev Mol Cell Biol, 2008, 9(2): pp. 162-76; all incorporated herein by reference) were selected. The selected candidate genes were uploaded to the Ingenuity Pathway Analysis 8.7 (IPA Ingenuity Systems®, Redwood City, Calif., USA, www.ingenuity.com) to find the upstream activators and inhibitors, and downstream target genes of the transcription regulators and the nuclear receptors. No filters were applied in IPA regarding species, tissues or cell lines, but an upper limit of 150 upstream and 150 downstream genes was defined. The SNPs linked to the extended selected list of 276 candidate genes were extracted from the dbSNP database (www.ncbi.nlm.nih.gov/projects/SNP, National Center for Biotechnology Information, U.S. National Library of Medicine, Bethesda), and matched with 469 SNPs on the Metabochip. These 469 SNPs (linked with 276 candidate genes) were uploaded to the web server FASTSNP (Yuan et al., Nucleic Acids Res, 2006, 34 (Web Server issue): pp. W635-41, incorporated herein by reference) to prioritize the SNPs that were most likely to have functional effect on the expression of the linked gene. According to a decision tree, each SNP was assigned a risk score between 0 and 5. Risk score 0 means that the SNP has no known effect (e.g. located in a downstream or upstream untranslated region, nearby the gene), and 5 means that the SNP has a functional effect (e.g. introduces a stop codon and hence premature translational termination). Basically all SNPs with risk score lower than 2 were discarded. Since several SNPs with risk score 2 or higher were linked to a single gene, we defined an upper limit of seven SNPs per gene. That was done by increasing the risk score claim one factor at the time, until the number of SNPs was at most seven. The result was a list of 190 SNPs.

### SNP Selection

Four different selections of SNPs were used in the analyses:
1. The ref-SNP selection—71 061 Metabochip SNPs assigned with a reference SNP ID (rs) with more than one SNP type among the 32 subjects. The ref-SNP selection was used to screen for SNPs that could be associated with HOMA2 IR.
2. The gene-SNP selection—a subset of 23 382 SNPs linked according to the dbSNP database with one or more genes present in the “gene expression dataset”. This resulted in 35 082 SNP and gene expression value (log₂-ratio) pairs, since several genes were represented with multiple probes on the HumanHT-12 Expression BeadChip. The gene-SNP selection was used to screen for pairs where the SNP was associated with the expression of the gene.
3. The candidate gene-SNP selection—the subset of 190 SNPs that according to the dbSNP database were linked with the genes in the candidate gene list (described above). This resulted in 364 SNP and gene expression value pairs. The candidate gene-SNP selection was used to screen for association between SNPs and HOMA2 IR, and associations between SNPs and gene expression.
4. The diabetes panel-SNP selection—a subset of 7 SNPs that according the dbSNP database were linked with genes coding for the proteins on the diabetes panel. This set of SNP selection was examined for association with the expression of proteins or genes of the diabetes panel. The SNPs were also tested for association with HOMA2 IR.

### Statistical Analyses

For all analyses a two-stage strategy was performed. In the first stage, analysis of variance (ANOVA) was performed to test the null hypothesis, whether there was no difference in either HOMA2 IR, gene expression (log₂-ratio), or protein concentration (logₑ-ratio) change between the genotypes. Genotype was used as covariate, and changes as response variables. P-values were adjusted for multiple testing using the Benjamini-Hochberg step up algorithm (Benjamini and Hochberg, Journal of the Royal Statistical Society. Series B (Methodological), 1995, 57(1): pp. 289-300, incorporated herein by reference) to control the false discovery rate (FDR). In the second stage, a one-sample, two-sided t-test was assigned to test if the change in HOMA2 IR, gene expression, or protein concentration, was different from zero for any of the genotypes. For the ref-SNP selection and the gene-SNP selection, the second stage was performed only for the 100 best ranked entries, according to the ANOVA p-values. Hence, eight Top100 lists were generated, one for each comparison, the ref-SNP selection and the gene-SNP selection separately (see Supplementary tables 1-8). Within these lists the t-test p-values were adjusted for multiple testing using the Benjamini-Hochberg step up algorithm.

Unsupervised hierarchical clustering analyses were performed, using Manhattan distance measures and complete linkage. PCA was performed with discrete data, where the three possible genotypes were represented by numerical values (0, 1, 2). Analyses were performed using the R statistical analysis framework (R Development Core Team, R: A Language and Environment for Statistical Computing, 2010; Available from: www.r-project.org, incorporated herein by reference).

Functional analysis to identify biological functions and diseases significantly associated with gene lists were performed using IPA 8.7 (Ingenuity). Since the Metabochip is custom made, biased by SNPs associated with metabolic and cardiovascular traits, a custom reference set was also used in all analyses. This was composed of all the 10 515 genes that according to the dbSNP database were linked to the 71 061 SNPs on the Metabochip. P-values were adjusted for multiple testing using the Benjamini-Hochberg step up algorithm.

### RESULTS

SNPs Associated with HOMA2 IR

Insulin resistance is a complex trait and the contribution of each single locus to the phenotype is small. To propose loci involved in the manifestation of this trait, the environmental homeostasis was challenged by introducing the subjects to two different diets. The responding change in HOMA2 IR for the four comparisons was related to SNPs in the ref-SNP selection. The biological relevance to insulin resistance was examined for all SNPs with FDR<0.2, a cut-off used in larger cohorts (>3000 subjects) earlier (Povel et al., Int J Obes (Lond), 2010, 34(5): pp. 840-5, incorporated herein by reference).

The change in HOMA2 IR during the AHC diet was associated (FDR<0.1) with four SNPs, with identical allele distribution between the subjects (FIG. 1A). The first SNP, rs16961756 (cgggccttcctcgccagcacctccattcct[a/g]aggctcacgtgggagagacagtgtggagag) (SEQ ID NO: 1) (Chr17:17359619, G→A) was located 126 base pairs (bp) upstream of a putative pseudogene (LOC100288179). This finding is supported by similar allele distribution in the closest neighboring SNP on the Metabochip, rs1242483 GAAGTTAAGAGTAAATAAAATAGTCA[C/T]GTTTGGGAGCATGAAGGAGCCGGC A (Chr17:17351675, T→C, P=0.002) (SEQ ID NO: 2)

The three other SNPs,

rs29095 (tccctcgaataaaggtgaaatttttaaaat[a/c]tcagtgaataggaatgtgcaaagactaag) (SEQ ID NO: 3) (Chr18:9957549),
rs7237794 (ctgcccctcccgacacagcacatacacaca[c/t]tgacgttttgctactacagcatatagcctt) (SEQ ID NO: 4) (Chr18:9951304), and
rs917688 (ttctctcatgcttaatatttggaactataa[a/c]gctaaaggccattgacgtagctaaaaatct) (SEQ ID NO: 5)(Chr18:9962736), (Chr18:9951304 . . . 9962736, C→A, T→C, and C→A, respectively) were closely linked, and rs7237794 and rs917688 were located in an intron region, and in the untranslated region of the 3′end of the gene vesicle-associated membrane protein-associated protein A, 33 kDa (VAPA), respectively. The 29 subjects homozygous for the consensus allele had an average downregulation of HOMA2 IR during the AHC diet (estimate of average change ( x̅)=−0.279, FDR=0.004), while the three remaining heterozygotes had an average upregulation ( x̅=1.000, FDR=0.098). This response to the AHC diet was only modestly reflected on the VAPA gene expression level. There was no change in HOMA2 IR among the homozygotes ( x̅=0.008, P=0.859), while among the heterozygotes there was a decrease ( x̅=−0.216, P=0.014).

Another association (FDR<0.02) was found between HOMA2 IR change during the AHC diet and the SNP rs10803976 (FIG. 1B) (Chr2:185428946, C→T)(CATTAA AAGCTATCATCTAACATTGC[C/T]TGGAGTGTTTATTTTTAAGTGCATA) (SEQ ID NO: 6), located 34 Kbp upstream of the nearest gene (zinc finger protein 804A). The 27 individuals homozygous for the consensus allele experienced an average decrease in HOMA2 IR during the AHC diet ( x̅=−0.311, FDR=0.004). The four heterozygotes experienced an average increase during the AHC diet ( x̅=0.800, FDR=0.103), and the response difference between the AHC ( x̅=0.800) diet and the BMC diet ( x̅=−0.275) was significant ( x̅=−1.075, FDR=0.048). Only one was homozygous for the alternative allele.

The same procedure was followed for the candidate gene-SNP list. HOMA2 IR change during the BMC diet was associated with the SNP rs6494711 (FIG. 1C) (aaaattgaggaaaatcccagaagatagagc[c/t]aaaagacaagagatgtaaaaatgcacaagg) (SEQ ID NO: 7) (FDR=0.047, Chr15:68374027, T→C). Those homozygous for this SNP had an average decrease in HOMA2 IR (TT, n=9, x̅=−0.644, P=0.004; CC, n=9, x̅=−0.422, P=0.003), while the heterozygotes had no significant change (CT, n=14, x̅=0.021, P=0.773). The SNP rs6494711 was located in an intron region of the transcription factor protein inhibitor of activated STAT-1 (PIAS1). The nearest neighbouring SNP on the Metabochip, rs1489595 AATTTTCTGTTTACACAAGTGATTCT[A/G]TAAGCAAACCAGGGTTCTCCATGGT (SEQ ID NO: 8) (Chr15:68377126, A→G, P=0.014), also located in an intron region of PIAS1, showed the same changes in HOMA2 IR among hetero- and homozygotes. The genotype specific changes were not reflected in the mRNA expression data of PIAS1.

SNP in the GIP Gene is Associated with PAI-1 Protein Concentration in Plasma

To screen for cis- and trans-regulating eQTLs that affected expression of genes central in pathogenesis of T2D, we tested the association between the SNPs in the diabetes panel-SNP and HOMA2 IR. Association was detected for the SNP rs2291726 (FIG. 1D) (Chr17:47039254, C→T)(TCTAGGGACACTTGAATCTTTTAATA[C/T]C TGAACCCCAAAAGCAGAGGGTACC) (SEQ ID NO: 9) and the protein concentration of PAI-1 in plasma. The SNP was located in an intron region of the gene coding for GIP. For the eight individuals homozygous for the consensus allele, the average protein concentration (logₑ-ratio) changed during the AHC diet differed significantly from the BMC diet ( x̅=−1.003, P=0.016). For the 21 heterozygotes and the three homozygous for the alternative allele there were only minor or no differences in PAI-1 concentration changes ( x̅=−0.037, P=0.695; x̅=−0.075, P=0.848, respectively). This suggests that nucleotide variation in GIP mRNA may have downstream effects on protein concentration of PAI-1. However, precaution has to be made interpreting this finding, since the deviation from HWE is significant (P=0.029).

SNPs Associated with HOMA2 IR Change are Related to Type 2 Diabetes

Since insulin resistance is manifested by numerous QTL, we wanted to explore how the genotype profile in each individual correlated with the change in HOMA2 IR in response to the diets. Heatmaps were generated showing hierarchical clustering of the ref-SNP selection Top100 SNPs associated with HOMA2 IR. SNPs were clustered according to allele distribution, and the subjects were sorted according to HOMA2 IR differences (increasing from left to right, FIG. 2A) in the comparison corresponding to the Top100 list. The genotype profiles for the subjects with the largest increase in HOMA2 IR during the AHC diet were notable (FIG. 2A, right). Some clusters of SNPs seem to have a dominant role influencing HOMA2 IR. The genotype profiles of the subjects with the largest decrease in HOMA2 IR during the BMC diet also differed distinctively from the rest of the subjects (FIG. 2B, left).

Since the subjects with the strongest increase in HOMA2 IR during the AHC diet had such a distinct genotype profile, these were suspected to be involved in our most significant associations between SNP and HOMA2 IR. To determine if this was due to technical artifacts we generated a dendrogram and a PCA plot (showing the three first principal components) based on allele information from the 71 061 SNPs to examine whether we had outlier individuals (FIG. 3). The figures suggest that there were two outliers, subjects 22 and 25, but neither of these contributed to the most significant associations between genotype and HOMA2 IR. However, those who did (especially subject 2, 12, 15, and 28) could not be considered as being outliers in these analysis, clustering well with the other subjects.

To explore functional and biological information about the SNPs that showed the highest association with HOMA2 IR, we assessed IPA's Functional Analysis. We extracted the genes that according the dbSNP database were linked to all the SNPs in the ref-SNP selection Top100 lists. We found 366 unique SNPs in the four lists, and these were linked with 150 unique genes. The gene set was significantly associated with T2D, displaying an FDR-value equal to 6.39×10⁻¹³ for the sum of all four Top100 lists (Table 1). The SNPs included in the ref-SNP selection Top100 lists were also associated with several traits that usually co-exist with insulin resistance, like cardiovascular disorder, hypertension, and immunological disorder.

### Genotype Specific Gene Expression Changes

To identify potential insulin resistance eQTLs, we matched the genes of the Top100 pairs from the gene-SNP lists for the four comparisons, with the genes related to insulin resistance in the literature, using the following search in PubMed (NCBI, NIH, USA): (“Diabetes Mellitus, Type 2”[MeSH] OR “Insulin resistance”[MeSH] OR “Hyperglycemia”[MeSH] OR “Insulin-Secreting Cells”[MeSH]). None of the pairs of SNPs and genes related to insulin resistance showed significant association between genotype and expression changes, but several genes showed significant genotype specific expression changes (FDR<0.05) in response to diet AHC and BMC (Table 2). This suggests that genotype is a considerable variable, contributing to interindividual gene expression variability.

### DISCUSSION

In this study we have defined a method to relate SNPs to phenotypic changes in response to an intervention, and applied this method to identify potential susceptibility loci for insulin resistance. The method should also be applicable on larger cohorts. We observed distinctive genotype profiles among strong responders to high and low glycemic load, concerning increase and decrease of insulin resistance, respectively. Several eQTL were found linked to genes related to insulin resistance, showing inter-genotype variability. On a limited number of subjects, we successfully applied statistical and bioinformatical methods new to this area of genetic research.

Our most significant finding is association of insulin resistance to VAPA, a protein previously shown to play a role in the vesicle budding and fusion events involving protein transport in cells (Weir et al., Biochem Biophys Res Commun, 2001, 286(3): pp. 616-21, incorporated herein by reference). GLUT4 is translocated to the surface of myocytes and adipocytes in response to insulin binding to its receptor. Various proteins control this GLUT4 translocation, including VAMP2 and syntaxin-4. VAPA interacts with both of these proteins in skeletal myoblasts, and is suggested to be a regulator of VAMP2 availability in insulin-dependent GLUT4 translocation (Foster, et al., Traffic, 2000, 1(6): pp. 512-21, incorporated herein by reference). The effect of insulin on GLUT4 translocation in monocytes is discussed, but there are indications that systemic insulin resistance is indicated by the presence of GLUT4 receptors on the monocyte surface (Mavros et al., Diabetes Res Clin Pract, 2009, 84(2): pp. 123-31, incorporated herein by reference. There is a strong association between variation in the SNPs rs29095, rs7237794, and rs917688 (FIG. 1A) and insulin resistance, modestly reflected in gene expression, showing that the subjects with decreased leukocyte expression of VAPA during the AHC diet experience an increased insulin resistance. This suggests that the chromosome region where these SNPs are located is a susceptibility locus concerning insulin resistance. It remains to be seen if leukocytes have a role as insulin target cells. The genetic variability in VAPA, eventually contributing to a change in insulin resistance, may be caused by stronger gene expression changes in cells traditionally regarded as insulin target cells. As far as we know, this is the first time an association is found between genetic variability in VAPA and insulin resistance. Earlier the SNP rs29066, located in the 3′UTR region of VAPA, between rs917688 and rs29095 has been found associated with bipolar disorder (Lohoff et al., J Neural Transm, 2008, 115(9): pp. 1339-45, incorporated herein by reference).

There are not many known genes regulated by the transcription factor PIAS1, but three of them, myogenin (MYOG) (Hsu et al., J Biol Chem, 2006, 281(44): pp. 33008-18, incorporated herein by reference), actin, alpha 2, smooth muscle, aorta (ACTA2, member of F-actin) (Kawai-Kowase et al., Mol Cell Biol, 2005, 25(18): pp. 8009-23, incorporated herein by reference), and cyclin-dependent kinase inhibitor 1A (CDKN1A) (Megidish et al., J Biol Chem, 2002, 277(10): pp. 8255-9, incorporated herein by reference) are all mediators of insulin induced signalling, shown in a variety of cells, including neutrophils, adipocytes, myocytes, pancreatic islet cells, and intestinal endocrine cells. (See Chodniewicz and Zhelev, Blood, 2003, 102(6): pp. 2251-8; Inoue et al., J Biol Chem, 2008, 283(30): pp. 21220-9; Kaneto et al., Diabetologia, 1999, 42(9): pp. 1093-7; Lim et al., Endocrinology, 2009, 150(12): pp. 5249-61; Sumitani et al., Endocrinology, 2002, 143(3): pp. 820-8; and Yoshizaki et al., Mol Cell Biol, 2007, 27(14): pp. 5172-83; all incorporated herein by reference.)

The association we found between the SNP rs6494711 and insulin resistance showed that homozygotes for both the consensus and the alternative allele had a decrease in insulin resistance during the BMC diet, but the heterozygotes had no significant change. However, the genotype specific change was not reflected in the mRNA expression data of PIAS1, but the effect of the transcription factors could be controlled by post-transcriptional activation. The effect may also be mediated through gene expression responses in other cells more insulin sensitive than leukocytes.

Increased PAI-1 concentration in the liver is associated with insulin resistance in mice (Takeshita et al., Metabolism, 2006, 55(11): pp. 1464-72, incorporated herein by reference), and loss of affinity between GIP and GIP-receptor affect localization of PAI-1 to mouse plasma (Hansotia et al., J Clin Invest, 2007, 117(1): pp. 143-52, incorporated herein by reference). Since GIP-secretion is stimulated by glucose, this could explain why genetic variation in the GIP gene was associated with changes in PAI-1 protein concentrations in plasma.

Today the recommendation of daily intake of carbohydrates in Norway is 50-60 E % (Utviklingen i norsk kosthold, Vol. 2008, Utviklingen i norsk kosthold 2008, Oslo: Direktoratet, 2008, 27 s, incorporated herein by reference). Such a high fraction will contribute to a high dietary glycemic load, unless considerable caution is taken to choose carbohydrate sources with low glycemic index. With precaution, regarding the small sample size, our results suggest that some individuals are sensitive to high glycemic load, which is shown by an increase in insulin resistance during high-carbohydrate dieting (AHC) (FIG. 2A). The same individuals have a distinct genotype profile for the SNPs most highly associated with changes in insulin resistance. Likewise, there are subjects that benefit more than others from low dietary glycemic load (FIG. 2B), also with a distinct genotype profile. The observation that a significant number of these SNPs are located in genes already associated with T2D and other traits related to insulin resistance strengthens our hypothesis that one could discern strong and weak responders to glycemic load, by their genotype profile. However, our contribution to identify these QTLs affecting insulin resistance should be corroborated in larger studies. Reliable personalized nutritional advice is something still far ahead, and the theme may also raise considerable ethical debate, but our results suggest that the population at large, but especially subjects predisposed to develop T2D, should be aware of the glycemic challenge that a diet with high glycemic load gives.

The use of genotyping data to link gene expression differences with phenotypes has increased markedly the last years. However, the use of genetic variation to stratify responses to a homeostatic challenge, like a diet intervention, has not been quite as common. The reason might be that the sample size required to gain significant results far exceeds what is easily manageable in an intervention study. We have shown that genotype is a source of interindividual variability in the response to a change in glycemic load, and suggest that genotype information can be integrated as an explanatory variable in microarray gene expression analysis.

Some obvious limitations need to be acknowledged in our study. What is already mentioned is the limited sample size. Whereas the study of average responses to a dietary intervention in a controlled cross-over study has produced robust findings (Arbo I, Brattbakk H R, Langaas M et al., A balanced macronutrient diet induces changes in a host of pro-inflammatory biomarkers, rendering a more healthy phenotype; a randomized cross-over trial, 2010, (manuscript submitted), incorporated herein by reference), dividing the subjects into two or three groups based on genotype inevitably decreases the statistical power. We nevertheless used reasonable criteria to declare associations of SNPs and eQTL (FDR<0.05), while acknowledging that of course in largest studies much more significant association confidence can be obtained. We considered various quality criteria of the SNPs that could account for aberrant behaviour in our statistical tests. One quality criterion concerns the Hardy-Weinberg equilibrium (HWE). In a population, deviation from HWE may be indicative of selective pressure, but because most genes are not under selection it can also be used as an indicator of problems in the genotyping procedure leading to bias in the observed allele frequencies (Greene et al., Lect Notes Comput Sci, 2010, 6023(LNCS): pp. 74-85, incorporated herein by reference). Another reason why the allele distribution might deviate from HWE is the relatively small sample size of the current study, making it vulnerable to biased selection of subjects. Nevertheless, except where noted, none of the SNPs for which we found associations showed a gross skewness in allele frequencies that would significantly violate HWE, and indeed all passed the SNP call quality criteria of the Genotyping V1.1.9 software (Illumina). To ascertain the genotyping and HWE quality of each individual SNP is challenging, so we did carefully consider these quality criteria when interpreting the results of individual SNPs.

Hierarchical clustering and PCA revealed two genetical outliers in our sample size (FIG. 3). Why these subjects deviate from the others is not known, but to re-analyse the data without these outliers would be a reasonable approach.

Leukocytes are an easy accessible source for transcriptome profiling, and an obvious choice to screen for inflammatory gene expression changes in response to food. However, the knowledge on insulin responsiveness is limited. The inflammatory properties of monocytes and macrophages are central in the development of insulin resistance in insulin target cells, like adipocytes and myocytes. But it is not known whether the established molecular mechanisms behind insulin resistance are the same in leukocytes. We have shown earlier that monocytes are insulin responsive in a dose dependent manner (Ingerid Arbo, Cathinka L Halle, Darshan Malik, et al. Insulin induces fatty acid desaturase expression in human monocytes, 2010, (manuscript submitted), incorporated herein by reference), inducing increased desaturase transcription. However, this does not guarantee that we can expect significant association between leukocyte gene expression and changes in insulin resistance, considering an earlier finding that gene expression profiles in leukocytes and adipocytes deviate (Brattbakk H R, Arbo I, Aagaard S, et al. Balanced caloric macronutrient composition downregulates immunological gene expression in human blood cells—adipose tissue diverges, 2010, (manuscript submitted), incorporated herein by reference). This demonstrates the need to investigate not only blood, but also additional parallel sampled biopsies of well established insulin target tissue, like adipose tissue.

Of the SNPs disclosed herein, it is seen that some are directly related to the genes for VAPA, Pias1 and GIP, while some are closely related thereto and can serve as “surrogate” markers. These SNPs are more specifically: rs16961756, rs1242483, rs29095, rs7237794, rs917688, rs6494711, rs1489595 and rs2291726.

The SNPs may serve as new markers of candidate QTL contributing to explain the genetic aspect of insulin resistance development. Also, VAPA and PIAS1 are new candidate genes involved in the molecular mechanisms behind insulin resistance. Finally, certain SNPs are candidate eQTL for plasma PAI-1 concentration, also related to insulin resistance. Our results have demonstrated the added value of incorporating genotype data in gene expression analysis to explain interindividual variability. A genotype profile of specific SNPs can distinguish weak and strong responders to glycemic load, with respect to insulin resistance. SNP typing may eventually be used to provide concrete dietary advice to persons genetically predisposed to T2D.

### REFERENCES

1. Tripathy, D. and A. O. Chavez, Defects in insulin secretion and action in the pathogenesis of type 2 diabetes mellitus. Curr Diab Rep, 2010. 10(3): p. 184-91.
2. Pagel-Langenickel, I., et al., The role of mitochondria in the pathophysiology of skeletal muscle insulin resistance. Endocr Rev, 2010. 31(1): p. 25-51.
3. Facchini, F. S., et al., Insulin resistance as a predictor of age-related diseases. J Clin Endocrinol Metab, 2001. 86(8): p. 3574-8.
4. McLaughlin, T., et al., Prevalence of insulin resistance and associated cardiovascular disease risk factors among normal weight, overweight, and obese individuals. Metabolism, 2004. 53(4): p. 495-9.
5. Meigs, J. B., Epidemiology of type 2 diabetes and cardiovascular disease: translation from population to prevention: the Kelly West award lecture 2009. Diabetes Care, 2010. 33(8): p. 1865-71.
6. Ludwig, D. S., The glycemic index: physiological mechanisms relating to obesity, diabetes, and cardiovascular disease. Jama, 2002. 287(18): p. 2414-23.
7. Lairon, D., et al., Nutrigenetics: links between genetic background and response to Mediterranean-type diets. Public Health Nutr, 2009. 12(9A): p. 1601-6.
8. Kaput, J., Nutrigenomics research for personalized nutrition and medicine. Curr Opin Biotechnol, 2008. 19(2): p. 110-20.
9. Martinez, J. A., et al., Genotype-dependent response to energy-restricted diets in obese subjects: towards personalized nutrition. Asia Pac J Clin Nutr, 2008. 17 Suppl 1: p. 119-22.
10. Goldberg, R. B., Cytokine and cytokine-like inflammation markers, endothelial dysfunction, and imbalanced coagulation in development of diabetes and its complications. J Clin Endocrinol Metab, 2009. 94(9): p. 3171-82.
11. Olefsky, J. M. and C. K. Glass, Macrophages, inflammation, and insulin resistance. Annu Rev Physiol, 2010. 72: p. 219-46.
12. Hotamisligil, G. S., Inflammation and metabolic disorders. Nature, 2006. 444(7121): p. 860-7.
13. Kushner, I., D. Samols, and M. Magrey, A unifying biologic explanation for “high-sensitivity” C-reactive protein and “low-grade” inflammation. Arthritis Care Res (Hoboken), 2010. 62(4): p. 442-6.
14. O'Keefe, J. H., N. M. Gheewala, and J. O. O'Keefe, Dietary strategies for improving post-prandial glucose, lipids, inflammation, and cardiovascular health. J Am Coll Cardiol, 2008. 51(3): p. 249-55.
15. Barclay, A. W., et al., Glycemic index, glycemic load, and chronic disease risk—a meta-analysis of observational studies. Am J Clin Nutr, 2008. 87(3): p. 627-37.
16. McKeown, N. M., et al., Carbohydrate nutrition, insulin resistance, and the prevalence of the metabolic syndrome in the Framingham Offspring Cohort. Diabetes Care, 2004. 27(2): p. 538-46.
17. Qi, L. and F. B. Hu, Dietary glycemic load, whole grains, and systemic inflammation in diabetes: the epidemiological evidence. Curr Opin Lipidol, 2007. 18(1): p. 3-8.
18. Voight, B. F., et al., Twelve type 2 diabetes susceptibility loci identified through large-scale association analysis. Nat Genet, 2010. 42(7): p. 579-89.
19. Kantartzis, K., et al., The DGAT2 gene is a candidate for the dissociation between fatty liver and insulin resistance in humans. Clin Sci (Lond), 2009. 116(6): p. 531-7.
20. Liu, P. H., et al., Genetic variants of TCF7L2 are associated with insulin resistance and related metabolic phenotypes in Taiwanese adolescents and Caucasian young adults. J Clin Endocrinol Metab, 2009. 94(9): p. 3575-82.
21. Palmer, N. D., et al., Association of protein tyrosine phosphatase 1B gene polymorphisms with measures of glucose homeostasis in Hispanic Americans: the insulin resistance atherosclerosis study (IRAS) family study. Diabetes, 2004. 53(11): p. 3013-9.
22. Richardson, D. K., et al., Association between variants in the genes for adiponectin and its receptors with insulin resistance syndrome (IRS)-related phenotypes in Mexican Americans. Diabetologia, 2006. 49(10): p. 2317-28.
23. Ruchat, S. M., et al., Associations between glucose tolerance, insulin sensitivity and insulin secretion phenotypes and polymorphisms in adiponectin and adiponectin receptor genes in the Quebec Family Study. Diabet Med, 2008. 25(4): p. 400-6.
24. Smith, S. R., et al., A promoter genotype and oxidative stress potentially link resistin to human insulin resistance. Diabetes, 2003. 52(7): p. 1611-8.
25. Jansen, R. C. and J. P. Nap, Genetical genomics: the added value from segregation. Trends Genet, 2001. 17(7): p. 388-91.
26. Schadt, E. E., et al., Genetics of gene expression surveyed in maize, mouse and man. Nature, 2003. 422(6929): p. 297-302.
27. Rodman, M. V. and L. Kruglyak, Genetics of global gene expression. Nat Rev Genet, 2006. 7(11): p. 862-72.
28. Wallace, T. M., J. C. Levy, and D. R. Matthews, Use and abuse of HOMA modeling. Diabetes Care, 2004. 27(6): p. 1487-95.
29. Matthews, D. R., et al., Homeostasis model assessment: insulin resistance and beta-cell function from fasting plasma glucose and insulin concentrations in man. Diabetologia, 1985. 28(7): p. 412-9.
30. Levy, J. C., D. R. Matthews, and M. P. Hermans, Correct homeostasis model assessment (HOMA) evaluation uses the computer program. Diabetes Care, 1998. 21(12): p. 2191-2.
31. Mojiminiyi, O. A. and N. A. Abdella, Effect of homeostasis model assessment computational method on the definition and associations of insulin resistance. Clin Chem Lab Med, 2010.
32. Illumina, GenomeStudio™ Genotyping Module v1.0 User Guide. 2008, Illumina, Inc: San Diego, Calif.
33. Wymann, M. P. and R. Schneiter, Lipid signalling in disease. Nat Rev Mol Cell Biol, 2008. 9(2): p. 162-76.
34. Yuan, H. Y., et al., FASTSNP: an always up-to-date and extendable service for SNP function analysis and prioritization. Nucleic Acids Res, 2006. 34(Web Server issue): p. W635-41.
35. Benjamini, Y. and Y. Hochberg, Controlling the False Discovery Rate: A Practical and Powerful Approach to Multiple Testing. Journal of the Royal Statistical Society. Series B (Methodological), 1995. 57(1): p. 289-300.
36. R Development Core Team, R: A Language and Environment for Statistical Computing. 2010; Available from: www.R-project.org.
37. Povel, C. M., et al., Glucose levels and genetic variants across transcriptional pathways: interaction effects with BMI. Int J Obes (Lond), 2010. 34(5): p. 840-5.
38. Weir, M. L., et al., VAP-A binds promiscuously to both v- and tSNAREs. Biochem Biophys Res Commun, 2001. 286(3): p. 616-21.
39. Foster, L. J., et al., A functional role for VAP-33 in insulin-stimulated GLUT4 traffic. Traffic, 2000. 1(6): p. 512-21.
40. Mavros, Y., D. Simar, and M. A. Singh, Glucose Tranporter-4 expression in monocytes: a systematic review. Diabetes Res Clin Pract, 2009. 84(2): p. 123-31.
41. Lohoff, F. W., et al., Association between polymorphisms in the vesicle-associated membrane protein-associated protein A (VAPA) gene on chromosome 18p and bipolar disorder. J Neural Transm, 2008. 115(9): p. 1339-45.
42. Hsu, Y. H., et al., Sumoylated SnoN represses transcription in a promoter-specific manner. J Biol Chem, 2006. 281(44): p. 33008-18.
43. Kawai-Kowase, K., et al., PIAS1 activates the expression of smooth muscle cell differentiation marker genes by interacting with serum response factor and class I basic helix-loop-helix proteins. Mol Cell Biol, 2005. 25(18): p. 8009-23.
44. Megidish, T., J. H. Xu, and C. W. Xu, Activation of p53 by protein inhibitor of activated Stat1 (PIAS1). J Biol Chem, 2002. 277(10): p. 8255-9.
45. Chodniewicz, D. and D. V. Zhelev, Novel pathways of F-actin polymerization in the human neutrophil. Blood, 2003. 102(6): p. 2251-8.
46. Inoue, N., et al., Cyclin-dependent kinase inhibitor, p21WAF1/CIP1, is involved in adipocyte differentiation and hypertrophy, linking to obesity, and insulin resistance. J Biol Chem, 2008. 283(30): p. 21220-9.
47. Kaneto, H., et al., Oxidative stress induces p21 expression in pancreatic islet cells: possible implication in beta-cell dysfunction. Diabetologia, 1999. 42(9): p. 1093-7.
48. Lim, G. E., et al., The rho guanosine 5′-triphosphatase, cell division cycle 42, is required for insulin-induced actin remodeling and glucagon-like peptide-1 secretion in the intestinal endocrine L cell. Endocrinology, 2009. 150(12): p. 5249-61.
49. Sumitani, S., et al., Akt1 and Akt2 differently regulate muscle creatine kinase and myogenin gene transcription in insulin-induced differentiation of C2C12 myoblasts. Endocrinology, 2002. 143(3): p. 820-8.
50. Yoshizaki, T., et al., Myosin 5a is an insulin-stimulated Akt2 (protein kinase Bbeta) substrate modulating GLUT4 vesicle translocation. Mol Cell Biol, 2007. 27(14): p. 5172-83.
51. Takeshita, Y., et al., Tumor necrosis factor-alpha-induced production of plasminogen activator inhibitor 1 and its regulation by pioglitazone and cerivastatin in a nonmalignant human hepatocyte cell line. Metabolism, 2006. 55(11): p. 1464-72.
52. Hansotia, T., et al., Extrapancreatic incretin receptors modulate glucose homeostasis, body weight, and energy expenditure. J Clin Invest, 2007. 117(1): p. 143-52.
53. Utviklingen i norsk kosthold. Vol. 2008, Utviklingen i norsk kosthold 2008., Oslo: Direktoratet, 2008. 27 s.
54. Greene, C. S., D. S. Himmelstein, and J. H. Moore, A Model Free Method to Generate Human Genetics Datasets with Complex Gene-Disease Relationships. Lect Notes Comput Sci, 2010. 6023(LNCS): p. 74-85.

All references cited herein, are hereby incorporated by reference. Sequences of the single nucleotide polymorphisms cited by the accession numbers herein, are hereby incorporated by reference and can be found at www.ncbi.nlm.nih.gov/sites/entrez or snpper.chip.org/bio, among other sites, using the accession numbers provided.

### Tables

TABLE 1
Biological functions and diseases related to the SNPs that
showed highest association with HOMA2 IR. The genes
linked to the SNPs in the ref-SNP selection Top 100 lists
were compared with the genes linked with the SNPs on the
Metabochip. Significantly enriched IPA defined functions and
diseases, according IPA's Functional Analysis, are listed
(FDR < 0.01). The table also shows the number of genes
related to the functions, linked with the SNPs in the ref-SNP
selection Top 100 lists for the AHC6-AHC0 and the BMC6-BMC0
comparisons separately.

| | All Top 100 lists | | AHC | BMC |
|---|---|---|---|---|
| Function Annotation | P-value | # genes | # genes | # genes |
| diabetes mellitus | 5.17E−13 | 63 | 15 | 23 |
| T2D | 6.39E−13 | 47 | 10 | 20 |
| endocrine system disorder | 1.68E−12 | 64 | — | 24 |
| metabolic disorder | 3.48E−12 | 67 | 16 | 24 |
| cardiovascular disorder | 6.46E−10 | 58 | 20 | 17 |
| hypertension | 8.65E−09 | 35 | 12 | — |
| atherosclerosis | 4.56E−08 | 38 | 13 | — |
| genetic disorder | 4.56E−08 | 102 | 27 | 35 |
| coronary artery disease | 5.01E−08 | 36 | 12 | 12 |
| T1D | 1.80E−07 | 33 | 11 | — |
| autoimmune disease | 8.59E−07 | 50 | 17 | 16 |
| immunological disorder | 1.96E−06 | 54 | 18 | 17 |
| amyotrophic lateral sclerosis | 1.53E−05 | 21 | 6 | 10 |
| progressive motor neuropathy | 1.68E−05 | 31 | 11 | — |
| Crohn's disease | 5.41E−05 | 29 | — | 11 |
| neurological disorder | 6.55E−05 | 66 | 19 | 26 |
| rheumatoid arthritis | 1.50E−04 | 35 | 13 | 10 |
| digestive system disorder | 2.48E−04 | 33 | — | 12 |
| inflammatory disorder | 5.16E−04 | 53 | 17 | — |
| Alzheimer's disease | 9.78E−04 | 23 | 6 | 10 |
| skeletal and muscular disorder | 1.80E−03 | 51 | 18 | 15 |
| rheumatic disease | 1.93E−03 | 36 | — | — |
| connective tissue disorder | 2.23E−03 | 37 | 14 | — |
| Parkinson's disease | 5.24E−03 | 17 | 7 | — |

TABLE 2
Pairs of associations between SNP and the gene expression values in nearest gene in
the gene-SNP selection Top100 lists. Pairs in which are included, the gene is related to
insulin resistance/T2D in at least one PubMed entry, and there is at least one significant
(FDR < 0.05, value typed in bold) genotype specific gene expression change (log₂-ratio) for
one of the four comparisons, and a GenTrainScore > 0.750.

| | Nearest | # PubMed | | | | | Nucleotide | Chitest100 | GenTrain |
|---|---|---|---|---|---|---|---|---|---|
| Comparison | Gene | citations | SNP | Frequency | Log₂-ratio | FDR | Substitution | (p) | Score |
| AHC6-AHC0 | PDZD2 | | rs283122 | | | | C → T | 0.099 | 0.866 |
| | | | CC | 1 | NA | NA | | | |
| | | | CT | 11 | 0.059 | 0.036 | | | |
| | | | TT | 18 | −0.031 | 0.086 | | | |
| | TPM1 | 1 | rs17752921 | | | | T → C | 0.291 | 0.850 |
| | | | TT | 27 | −0.123 | <0.001 | | | |
| | | | CT | 3 | 0.122 | 0.378 | | | |
| | LPA | 9 | rs6415084 | | | | T → C | 0.055 | 0.838 |
| | | | CC | 6 | 0.016 | 0.801 | | | |
| | | | CT | 18 | 0.019 | 0.307 | | | |
| | | | TT | 6 | −0.127 | 0.039 | | | |
| | NME1 | 1 | rs2302254 | | | | C → T | 0.234 | 0.769 |
| | | | CC | 23 | −0.058 | 0.108 | | | |
| | | | CT | 7 | 0.126 | 0.042 | | | |
| | ADRA1A | 1 | rs4732874 | | | | T → C | 0.462 | 0.879 |
| | | | CC | 17 | −0.070 | 0.031 | | | |
| | | | CT | 10 | 0.070 | 0.108 | | | |
| | | | TT | 3 | −0.027 | 0.431 | | | |
| | ARHGEF11 | 3 | rs822570 | | | | T → C | 0.030 | 0.927 |
| | | | CC | 11 | −0.061 | 0.417 | | | |
| | | | CT | 12 | 0.229 | 0.005 | | | |
| | | | TT | 7 | 0.012 | 0.910 | | | |
| | SIK3 | 1 | rs888246 | | | | C → T | 0.463 | 0.838 |
| | | | CC | 25 | −0.084 | 0.130 | | | |
| | | | CT | 5 | −0.429 | 0.031 | | | |
| | TMEM195 | 1 | rs7781413 | | | | A → G | 0.943 | 0.869 |
| | | | AA | 21 | −0.036 | 0.021 | | | |
| | | | AG | 8 | 0.057 | 0.280 | | | |
| | | | GG | 1 | NA | NA | | | |
| | ADCY9 | 1 | rs2532007 | | | | G → A | 0.424 | 0.856 |
| | | | AA | 24 | 0.038 | 0.312 | | | |
| | | | AG | 6 | −0.226 | 0.184 | | | |
| | SLC2A1 | 6 | rs751210 | | | | G → A | 0.051 | 0.808 |
| | | | AA | 1 | NA | NA | | | |
| | | | AG | 14 | −0.110 | 0.328 | | | |
| | | | GG | 15 | −0.513 | 0.002 | | | |
| | PLTP | 9 | rs435306 | | | | T → G | 0.495 | 0.847 |
| | | | AA | 19 | −0.039 | 0.048 | | | |
| | | | AC | 9 | 0.047 | 0.180 | | | |
| | | | CC | 2 | NA | NA | | | |
| | | | rs378114 | | | | C → T | 0.495 | 0.904 |
| | | | AA | 2 | NA | NA | | | |
| | | | AG | 9 | 0.047 | 0.180 | | | |
| | | | GG | 19 | −0.039 | 0.048 | | | |
| BMC6-BMC0 | CAMTA1 | 1 | rs6577435 | | | | C → A | 0.440 | 0.873 |
| | | | AA | 1 | NA | NA | | | |
| | | | AC | 10 | 0.105 | 0.232 | | | |
| | | | CC | 21 | −0.117 | 0.009 | | | |
| | CACNA1G | 1 | rs989128 | | | | G → A | 0.012 | 0.785 |
| | | | AA | 6 | −0.017 | 0.513 | | | |
| | | | AG | 10 | −0.081 | 0.028 | | | |
| | | | GG | 16 | 0.013 | 0.323 | | | |
| | SULF2 | 1 | rs6125103 | | | | C → T | 0.203 | 0.837 |
| | | | CC | 22 | 0.210 | 0.011 | | | |
| | | | CT | 9 | −0.128 | 0.220 | | | |
| | | | TT | 1 | NA | NA | | | |
| BMC6-AHC6 | ITGAV | 1 | rs3738919 | | | | C → A | 0.595 | 0.925 |
| | | | AA | 8 | 0.515 | 0.032 | | | |
| | | | AC | 14 | −0.018 | 0.764 | | | |
| | | | CC | 10 | −0.060 | 0.657 | | | |
| | SULF2 | 1 | rs11699888 | | | | C → T | 0.421 | 0.862 |
| | | | CC | 27 | −0.054 | 0.294 | | | |
| | | | CT | 5 | 0.359 | 0.039 | | | |

### FIGURE LEGENDS

FIG. 1 Changes in HOMA2 IR or protein concentration (log₂-ratio), separately for each comparison, and genotype for the SNPs indicated. GenTrain score>0.73 for all SNPs (A-D). The SNP rs2291726 (D) deviated from HWE (P<0.05).

FIG. 2 Unsupervised hierarchical clustering (Manhattan distance measures, complete linkage) of the Top100 SNPs (rows) associated with change in HOMA2 IR in response to A) the AHC diet, and B) the BMC diet. The subjects (columns) are sorted by HOMA2 IR change, increasing from left to right. SNPs within the right hand side brackets of the heatmaps are identified in the ref-SNP selection Top100 lists in Supplementary table 1-4.

FIG. 3 A) Hierarchical clustering showing distance between subjects based on genotype information from the 71 061 SNPs (Manhattan distance measures, complete linkage). B) PCA plot based on the same data, showing the 3 first principal components.

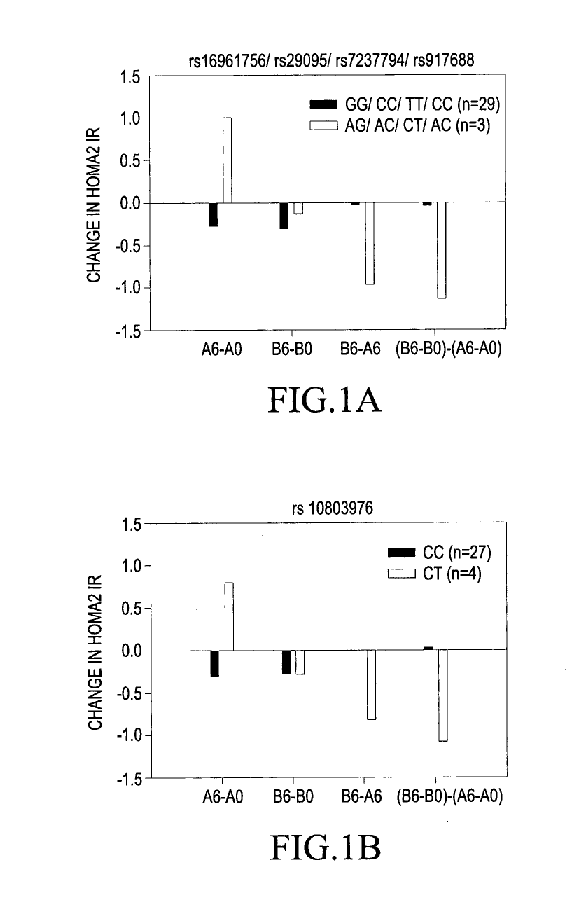

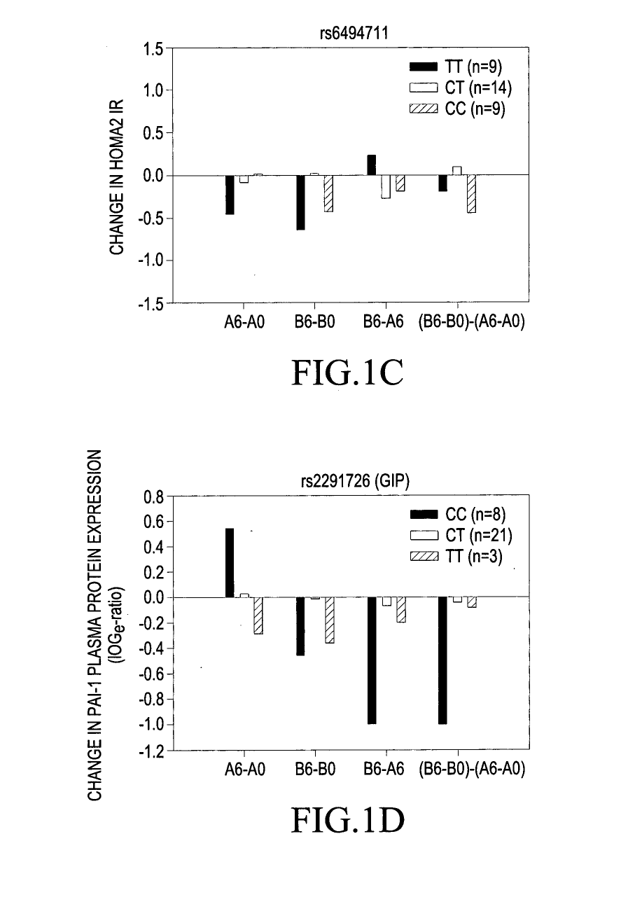

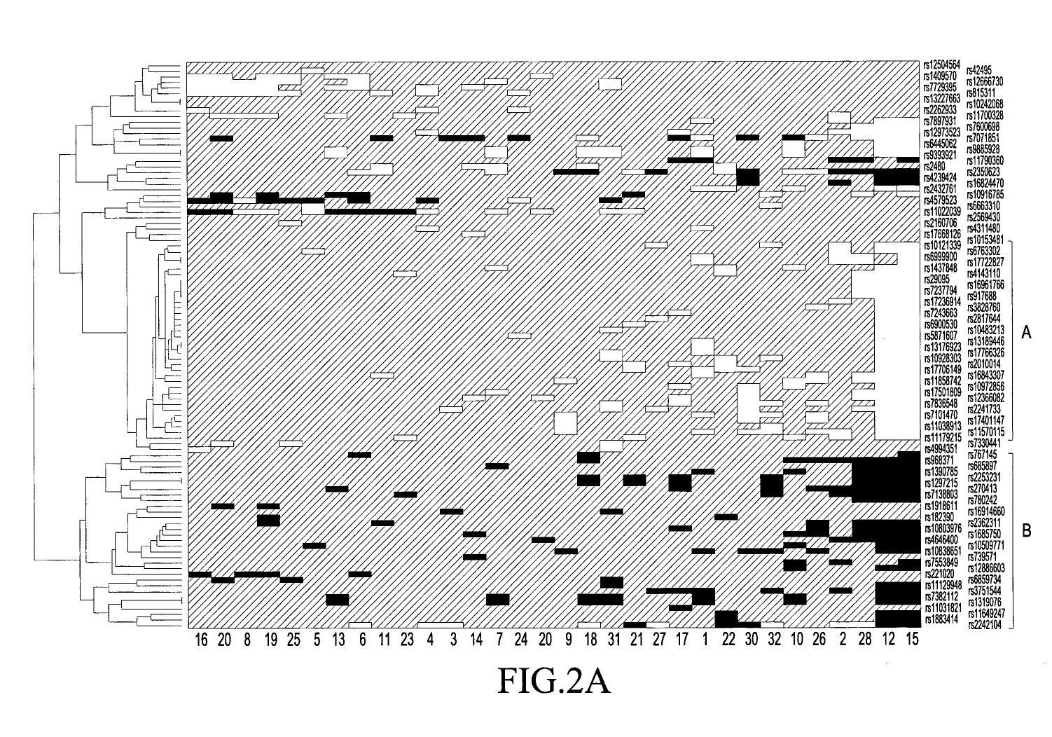

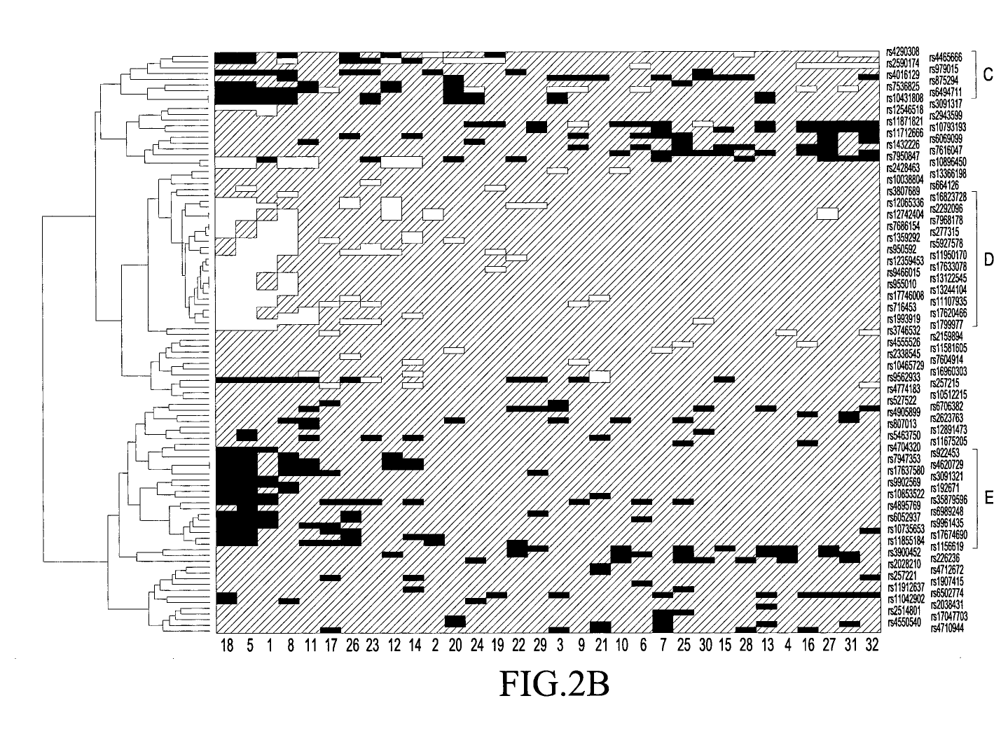

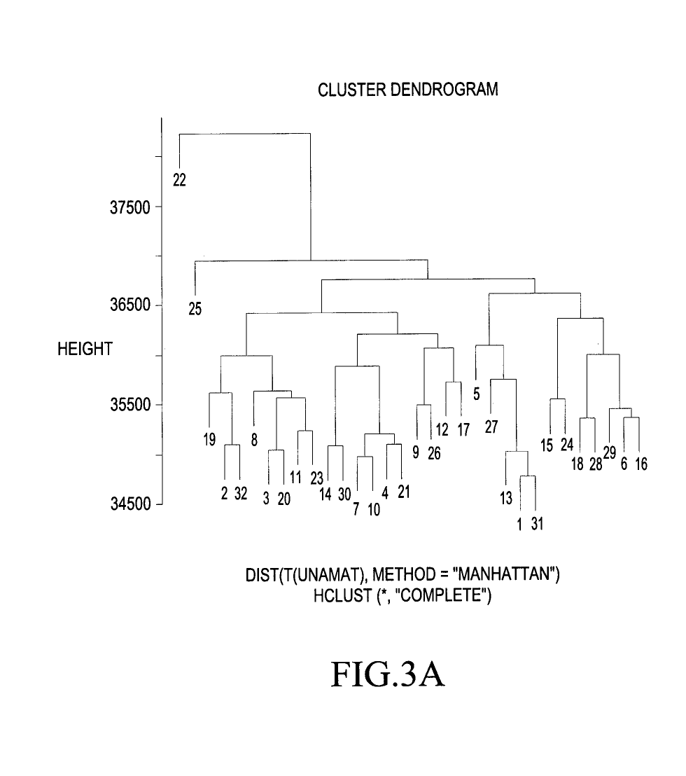

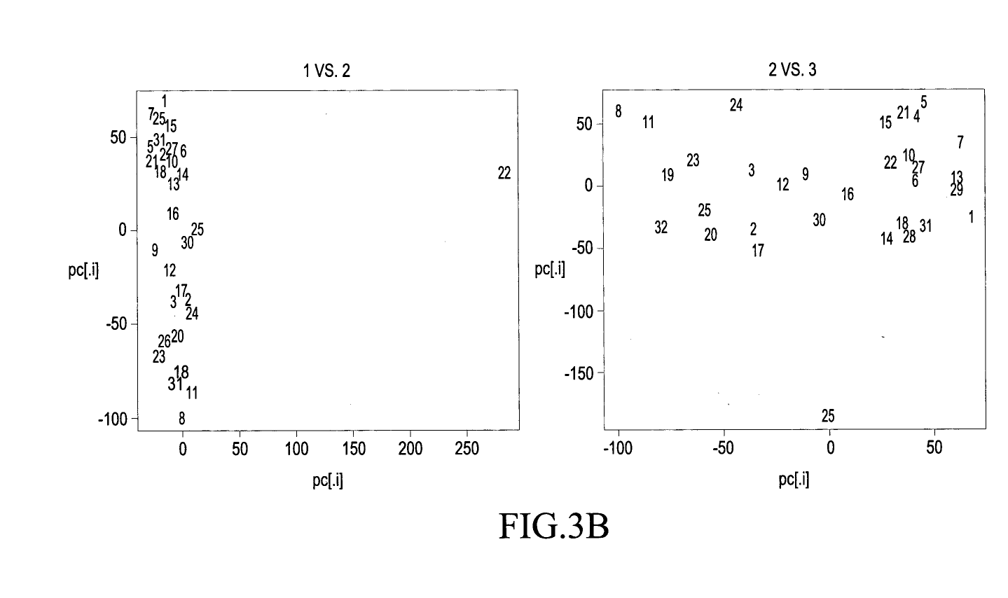

SUPPLEMENTARY TABLE 1
Ref-SNP selection Top 100 list, AHC6-AHC0

| SNP | Nearest Gene | ANOVA p-value | GenTrain Score | ChiTest100 | Cluster |
|---|---|---|---|---|---|
| rs16961756 | | <0.001 | 0.736 | 0.421 | B |
| rs29095 | VAPA | <0.001 | 0.875 | 0.657 | B |
| rs7237794 | VAPA | <0.001 | 0.843 | 0.688 | B |
| rs917688 | | <0.001 | 0.738 | 0.657 | B |
| rs10803976 | | <0.001 | 0.885 | 0.463 | A |
| rs780242 | | <0.001 | 0.904 | 0.091 | A |
| rs16914660 | ANK3 | <0.001 | 0.842 | 0.667 | A |
| rs7600698 | | <0.001 | 0.507 | 0.362 | |
| rs17236914 | | <0.001 | 0.845 | 0.072 | B |
| rs6753302 | EPAS1 | <0.001 | 0.837 | <0.001 | B |
| rs11858742 | ZWILCH | <0.001 | 0.832 | 0.354 | B |
| rs11031821 | | <0.001 | 0.901 | 0.152 | A |
| rs6445062 | | <0.001 | 0.877 | 0.067 | |
| rs4646400 | PEMT | <0.001 | 0.798 | 0.463 | A |
| rs3828760 | FAM46A | <0.001 | 0.885 | 0.004 | B |
| rs1390785 | GALNTL6 | <0.001 | 0.901 | 0.742 | A |
| rs10509771 | CCDC147 | <0.001 | 0.891 | 0.004 | A |
| rs2480 | | <0.001 | 0.912 | 0.511 | |
| rs968371 | CSMD1 | <0.001 | 0.811 | 0.670 | A |
| rs13176923 | | <0.001 | 0.751 | 0.657 | B |
| rs13189446 | | <0.001 | 0.824 | 0.688 | B |
| rs7243663 | L3MBTL4 | <0.001 | 0.771 | 0.688 | B |
| rs17501809 | C9orf46 | <0.001 | 0.804 | 0.352 | B |
| rs11038913 | AMBRA1 | <0.001 | 0.841 | 0.163 | B |
| rs17401147 | | <0.001 | 0.885 | 0.073 | B |
| rs42495 | SEMA5A | <0.001 | 0.915 | 0.857 | |
| rs10972856 | | <0.001 | 0.777 | 0.234 | B |
| rs11570115 | MYBPC3 | <0.001 | 0.888 | 0.144 | B |
| rs4579523 | | <0.001 | 0.802 | <0.001 | |
| rs7101470 | C11orf49 | <0.001 | 0.917 | 0.655 | B |
| rs10838651 | C11orf49 | <0.001 | 0.903 | 0.655 | A |
| rs16843307 | | <0.001 | 0.936 | 0.073 | B |
| rs12666730 | | <0.001 | 0.897 | 0.857 | |
| rs2362311 | ABCA13 | <0.001 | 0.826 | 0.072 | A |
| rs11179215 | TRHDE | <0.001 | 0.898 | 0.295 | B |
| rs1883414 | LOC100294320 | 0.001 | 0.929 | 0.063 | A |
| rs10121339 | | 0.001 | 0.819 | 0.421 | B |
| rs4143110 | | 0.001 | 0.935 | 0.424 | B |
| rs12973523 | FCER2 | 0.001 | 0.771 | 0.425 | |
| rs12486603 | MYRIP | 0.001 | 0.914 | 0.011 | A |
| rs7071851 | PTCHD3 | 0.001 | 0.893 | 0.523 | |
| rs2817644 | | 0.001 | 0.816 | 0.574 | B |
| rs6902530 | | 0.001 | 0.784 | 0.657 | B |
| rs10483213 | CENPM | 0.001 | 0.767 | 0.495 | B |
| rs11700328 | ANGPT4 | 0.001 | 0.784 | 0.425 | |
| rs16824470 | | 0.001 | 0.911 | 0.863 | |
| rs815811 | C2orf61 | 0.001 | 0.795 | 0.144 | |
| rs2350623 | C22orf9 | 0.001 | 0.889 | 0.285 | |
| rs10242065 | | 0.001 | 0.830 | 0.018 | |
| rs13227663 | | 0.001 | 0.860 | 0.027 | |
| rs7836548 | | 0.001 | 0.864 | 0.109 | B |
| rs11790360 | | 0.001 | 0.811 | 0.474 | |
| rs2262933 | SMYD3 | 0.001 | 0.863 | 0.526 | |
| rs685897 | | 0.001 | 0.835 | 0.846 | A |
| rs4239424 | | 0.001 | 0.712 | 0.001 | |
| rs1409570 | | 0.001 | 0.695 | 0.185 | |
| rs7897931 | | 0.001 | 0.836 | 0.339 | |
| rs11022039 | | 0.001 | 0.873 | 0.523 | |
| rs7553849 | PRDM16 | 0.001 | 0.750 | 0.079 | A |
| rs2569430 | CTU1 | 0.001 | 0.778 | 0.015 | |
| rs4994351 | ZNF331 | 0.001 | 0.741 | 0.049 | |
| rs12366082 | DSCAML1 | 0.001 | 0.746 | 0.290 | B |
| rs1918611 | ABCA13 | 0.001 | 0.872 | 0.268 | A |
| rs6871607 | | 0.001 | 0.865 | 0.574 | B |
| rs11649247 | WWOX | 0.001 | 0.707 | 0.462 | A |
| rs17722827 | | 0.001 | 0.754 | 0.421 | B |
| rs17766326 | SLC25A21 | 0.001 | 0.863 | 0.495 | B |
| rs5999900 | | 0.001 | 0.713 | 0.354 | B |
| rs1885750 | | 0.001 | 0.724 | 0.162 | A |
| rs12504564 | TMEM144 | 0.001 | 0.826 | 0.058 | |
| rs182390 | | 0.001 | 0.896 | 0.495 | A |
| rs10153481 | ZNF709 | 0.001 | 0.682 | 0.002 | |
| rs17706149 | NUP35 | 0.001 | 0.876 | 0.290 | B |
| rs7138803 | | 0.001 | 0.854 | 0.041 | A |
| rs10928303 | | 0.001 | 0.858 | 0.291 | B |
| rs1437848 | GALNTL6 | 0.001 | 0.907 | 0.354 | B |
| rs2010014 | | 0.001 | 0.879 | 0.352 | B |
| rs221020 | PAK7 | 0.001 | 0.915 | 0.667 | A |
| rs10916785 | | 0.001 | 0.741 | 0.001 | |
| rs270413 | BMP6 | 0.001 | 0.826 | 0.146 | A |
| rs1297215 | NRIP1 | 0.001 | 0.910 | 0.064 | A |
| rs2253231 | NRIP1 | 0.001 | 0.912 | 0.064 | A |
| rs7729395 | | 0.001 | 0.876 | 0.185 | |
| rs2242104 | VLDLR | 0.001 | 0.894 | 0.189 | A |
| rs767145 | | 0.001 | 0.808 | 0.835 | A |
| rs739571 | | 0.001 | 0.901 | 0.336 | A |
| rs17668126 | | 0.001 | 0.797 | 0.336 | |
| rs2432761 | FARS2 | 0.002 | 0.913 | 0.672 | |
| rs3751544 | MEIS2 | 0.002 | 0.885 | 0.425 | A |
| rs6663310 | | 0.002 | 0.756 | 0.001 | |
| rs4311480 | FILIP1 | 0.002 | 0.890 | 0.830 | |
| rs2241733 | PLXNA4 | 0.002 | 0.763 | 0.234 | B |
| rs2160706 | | 0.002 | 0.836 | 0.225 | |
| rs11129948 | | 0.002 | 0.827 | 0.600 | A |
| rs6859734 | ADAMTS19 | 0.002 | 0.863 | <0.001 | A |
| rs7380441 | ADAMTS19 | 0.002 | 0.805 | <0.001 | |
| rs1319075 | | 0.002 | 0.820 | 0.057 | A |
| rs7382112 | | 0.002 | 0.906 | 0.019 | A |
| rs9393921 | | 0.002 | 0.880 | 0.021 | |
| rs9885928 | | 0.002 | 0.820 | 0.057 | |

SUPPLEMENTARY TABLE 2
Ref-SNP selection Top100 list, BMC6-BMC0

| | | | GenTrain | | |
|---|---|---|---|---|---|
| SNP | Nearest Gene | ANOVA p-value | Score | ChiTest100 | Cluster |
| rs2623763 | | <0.001 | 0.670 | 0.088 | |
| rs4712572 | CDKAL1 | <0.001 | 0.760 | 0.062 | |
| rs1993919 | STAB2 | <0.001 | 0.881 | 0.144 | D |
| rs7536825 | KIF26B | <0.001 | 0.846 | 0.131 | C |
| rs16960303 | CDH13 | <0.001 | 0.903 | 0.142 | |
| rs9902569 | | <0.001 | 0.922 | 0.667 | E |
| rs13244124 | CDK14 | <0.001 | 0.819 | 0.018 | D |
| rs6052937 | SLC23A2 | <0.001 | 0.884 | <0.001 | E |
| rs12359453 | | <0.001 | 0.789 | 0.742 | D |
| rs6069099 | | <0.001 | 0.837 | 0.285 | |
| rs17620466 | | <0.001 | 0.738 | 0.495 | D |
| rs9961435 | | <0.001 | 0.788 | 0.162 | E |
| rs875294 | | <0.001 | 0.815 | 0.234 | C |
| rs4465666 | | <0.001 | 0.606 | <0.001 | C |
| rs11912637 | | <0.001 | 0.883 | 0.268 | |
| rs10431808 | CLN6 | <0.001 | 0.822 | 0.888 | |
| rs6494711 | PIAS1 | <0.001 | 0.924 | 0.888 | C |
| rs4895769 | | <0.001 | 0.887 | 0.225 | E |
| rs11855184 | DMXL2 | <0.001 | 0.897 | 0.049 | E |
| rs10512215 | | <0.001 | 0.899 | 0.526 | |
| rs13122545 | FAM190A | <0.001 | 0.892 | 0.291 | D |
| rs9466015 | | <0.001 | 0.917 | 0.742 | D |
| rs955010 | FAM190A | <0.001 | 0.916 | 0.290 | D |
| rs4704320 | IQGAP2 | <0.001 | 0.877 | 0.399 | E |
| rs3807689 | MAGI2 | <0.001 | 0.818 | 0.672 | D |
| rs4650540 | | <0.001 | 0.930 | 0.260 | |
| rs13356198 | CHSY3 | <0.001 | 0.875 | 0.440 | |
| rs6463750 | | <0.001 | 0.855 | 0.027 | |
| rs257215 | | <0.001 | 0.875 | 0.009 | |
| rs257221 | | <0.001 | 0.858 | 0.009 | |
| rs3746532 | LOC100287002 | 0.001 | 0.673 | 0.508 | |
| rs11107935 | NAV3 | 0.001 | 0.916 | 0.352 | D |
| rs1359292 | CCDC30 | 0.001 | 0.737 | 0.290 | D |
| rs10896450 | | 0.001 | 0.838 | 0.595 | |
| rs4620729 | | 0.001 | 0.802 | 0.318 | E |
| rs7947353 | | 0.001 | 0.902 | 0.595 | E |
| rs6706382 | | 0.001 | 0.877 | 0.244 | |
| rs17047703 | TGFB2 | 0.001 | 0.801 | 0.871 | |
| rs11581605 | TGFB2 | 0.001 | 0.924 | 0.871 | |
| rs7950547 | | 0.001 | 0.840 | 0.828 | |
| rs10793139 | | 0.001 | 0.824 | 0.799 | |
| rs12742404 | CAMSAP1L1 | 0.001 | 0.951 | 0.073 | D |
| rs2292096 | CAMSAP1L1 | 0.001 | 0.902 | 0.073 | D |
| rs2514801 | CDH17 | 0.001 | 0.875 | 0.349 | |
| rs2338545 | PLB1 | 0.001 | 0.810 | 0.440 | |
| rs11675205 | TCF7L1 | 0.001 | 0.918 | 0.614 | |
| rs4710944 | CDKAL1 | 0.001 | 0.884 | 0.244 | |
| rs627522 | ZNF708 | 0.001 | 0.894 | 0.724 | |
| rs4774183 | | 0.001 | 0.507 | <0.001 | |
| rs12065336 | | 0.001 | 0.728 | 0.290 | D |
| rs950692 | GPR98 | 0.001 | 0.751 | 0.234 | D |
| rs11712666 | VGLL4 | 0.001 | 0.733 | 0.131 | |
| rs12546518 | GRHL2 | 0.001 | 0.808 | 0.152 | |
| rs3091317 | | 0.001 | 0.903 | 0.899 | |
| rs3091321 | CCL7 | 0.001 | 0.901 | 0.863 | |
| rs12891473 | SRP54 | 0.001 | 0.698 | 0.001 | |
| rs6989246 | MYOM2 | 0.001 | 0.815 | 0.943 | E |
| rs11871821 | | 0.001 | 0.685 | <0.001 | |
| rs17746008 | PHLPP1 | 0.001 | 0.895 | 0.502 | D |
| rs1799977 | MLH1 | 0.001 | 0.936 | 0.614 | |
| rs807013 | | 0.001 | 0.693 | 0.003 | |
| rs1907415 | | 0.001 | 0.935 | 0.526 | |
| rs7616047 | | 0.001 | 0.886 | 0.146 | |
| rs10735653 | | 0.001 | 0.829 | <0.001 | E |
| rs2590174 | | 0.001 | 0.861 | 0.924 | C |
| rs35879596 | GRAMD1A | 0.001 | 0.887 | 0.440 | E |
| rs4290308 | | 0.001 | 0.822 | 0.042 | C |
| rs1432226 | THSD7B | 0.001 | 0.804 | 0.075 | |
| rs11042902 | MRVI1 | 0.001 | 0.872 | 0.459 | |
| rs979015 | | 0.001 | 0.807 | 0.137 | C |
| rs4555526 | | 0.001 | 0.910 | 0.017 | |
| rs2425463 | CHD6 | 0.001 | 0.821 | 0.036 | |
| rs17637580 | LARS2 | 0.001 | 0.822 | 0.015 | E |
| rs10465729 | | 0.001 | 0.833 | 0.011 | |
| rs10038804 | UGT3A2 | 0.001 | 0.865 | 0.320 | |
| rs2038431 | ZFP64 | 0.001 | 0.726 | 0.943 | |
| rs7604914 | FAM82A1 | 0.001 | 0.898 | 0.916 | |
| rs3900452 | | 0.001 | 0.762 | 0.195 | |
| rs4016189 | | 0.001 | 0.890 | 0.195 | C |
| rs2159894 | | 0.001 | 0.773 | 0.672 | |
| rs17674590 | | 0.001 | 0.811 | 0.295 | E |
| rs1156619 | | 0.001 | 0.912 | 0.244 | |
| rs922453 | | 0.001 | 0.882 | 0.667 | E |
| rs654126 | CSMD1 | 0.001 | 0.909 | 0.108 | |
| rs6927578 | PARK2 | 0.001 | 0.815 | 0.463 | D |
| rs11950170 | | 0.001 | 0.849 | 0.421 | D |
| rs16823728 | C2orf83 | 0.001 | 0.776 | 0.688 | D |
| rs17633078 | KATNAL1 | 0.001 | 0.883 | 0.502 | D |
| rs277315 | | 0.001 | 0.736 | 0.502 | D |
| rs9562933 | | 0.001 | 0.864 | 0.042 | |
| rs716453 | PPAPDC1A | 0.001 | 0.731 | 0.657 | D |
| rs7686154 | | 0.001 | 0.865 | 0.023 | D |
| rs7968178 | | 0.001 | 0.911 | 0.657 | D |
| rs226236 | LASP1 | 0.001 | 0.804 | 0.177 | |
| rs2943599 | | 0.001 | 0.869 | 0.318 | |
| rs10853522 | | 0.001 | 0.906 | 0.924 | E |
| rs192671 | CCDC50 | 0.001 | 0.881 | 0.441 | E |
| rs6502774 | TUSC5 | 0.001 | 0.808 | 0.268 | |
| rs4905899 | EML1 | 0.001 | 0.788 | 0.001 | |
| rs2028210 | AMPH | 0.001 | 0.890 | 0.672 | |

SUPPLEMENTARY TABLE 3
Ref-SNP selection Top100 list, BMC6-AHC6

| | Nearest | ANOVA p- | GenTrain | |
|---|---|---|---|---|
| SNP | Gene | value | Score | ChiTest100 |
| rs12304001 | | <0.001 | 0.826 | 0.399 |
| rs17236914 | | <0.001 | 0.845 | 0.072 |
| rs6753302 | EPAS1 | <0.001 | 0.837 | <0.001 |
| rs16916966 | | <0.001 | 0.878 | 0.001 |
| rs1556260 | USF1 | <0.001 | 0.918 | 0.005 |
| rs7597683 | | <0.001 | 0.814 | 0.094 |
| rs7160372 | | <0.001 | 0.804 | 0.267 |
| rs7196505 | | <0.001 | 0.849 | 0.225 |
| rs1996806 | RGS7 | <0.001 | 0.881 | 0.462 |
| rs11558471 | SLC30A8 | <0.001 | 0.906 | 0.846 |
| rs9296579 | | <0.001 | 0.808 | <0.001 |
| rs12468863 | KCNK3 | <0.001 | 0.834 | 0.295 |
| rs1275941 | | <0.001 | 0.855 | 0.549 |
| rs3739081 | | <0.001 | 0.940 | 0.549 |
| rs6859734 | ADAMTS19 | <0.001 | 0.863 | <0.001 |
| rs7380441 | ADAMTS19 | <0.001 | 0.805 | <0.001 |
| rs10220965 | | <0.001 | 0.756 | 0.109 |
| rs1488666 | | <0.001 | 0.693 | 0.506 |
| rs2781792 | | <0.001 | 0.741 | 0.185 |
| rs17069214 | | <0.001 | 0.931 | 0.203 |
| rs17245857 | | <0.001 | 0.827 | 0.055 |
| rs7553849 | PRDM16 | <0.001 | 0.750 | 0.079 |
| rs2235642 | IFT140 | <0.001 | 0.694 | 0.667 |
| rs3758376 | SEC61A2 | <0.001 | 0.836 | 0.116 |
| rs2305413 | CHRNA1 | <0.001 | 0.909 | 0.424 |
| rs12903587 | CHD2 | <0.001 | 0.853 | 0.393 |
| rs2062096 | | <0.001 | 0.938 | <0.001 |
| rs12467466 | CENPA | <0.001 | 0.754 | 0.362 |
| rs3802177 | SLC30A8 | <0.001 | 0.932 | 0.672 |
| rs11179215 | TRHDE | <0.001 | 0.898 | 0.295 |
| rs2399786 | NUDT5 | <0.001 | 0.910 | 0.659 |
| rs6744164 | | 0.001 | 0.812 | 0.421 |
| rs968371 | CSMD1 | 0.001 | 0.811 | 0.670 |
| rs13266634 | SLC30A8 | 0.001 | 0.881 | 0.914 |
| rs7855478 | MORN5 | 0.001 | 0.692 | 0.109 |
| rs12424799 | | 0.001 | 0.773 | 0.080 |
| rs12891948 | | 0.001 | 0.754 | 0.320 |
| rs17801467 | | 0.001 | 0.840 | 0.290 |
| rs929269 | ENDOU | 0.001 | 0.727 | 0.421 |
| rs281385 | MAMSTR | 0.001 | 0.721 | 0.290 |
| rs12964419 | | 0.001 | 0.919 | 0.871 |
| rs2274305 | DCDC2 | 0.001 | 0.862 | 0.290 |
| rs488078 | | 0.001 | 0.869 | 0.548 |
| rs10947465 | | 0.001 | 0.805 | 0.393 |
| rs17484283 | | 0.001 | 0.785 | 0.001 |
| rs12982980 | ZNF468 | 0.001 | 0.638 | 0.022 |
| rs4466385 | C8orf34 | 0.001 | 0.881 | 0.067 |
| rs17736747 | | 0.001 | 0.849 | 0.295 |
| rs4644227 | C8orf34 | 0.001 | 0.863 | 0.511 |
| rs17635121 | | 0.001 | 0.934 | 0.019 |
| rs1674091 | DTX1 | 0.001 | 0.689 | 0.080 |
| rs472972 | POLN | 0.001 | 0.825 | 0.421 |
| rs1487775 | | 0.001 | 0.862 | 0.295 |
| rs2072844 | | 0.001 | 0.872 | 0.062 |
| rs1861699 | | 0.001 | 0.874 | 0.587 |
| rs3788464 | SYN3 | 0.001 | 0.873 | 0.657 |
| rs942024 | | 0.001 | 0.752 | 0.672 |
| rs12973523 | FCER2 | 0.001 | 0.771 | 0.425 |
| rs11964281 | ESR1 | 0.001 | 0.771 | 0.421 |
| rs7025024 | | 0.001 | 0.795 | 0.549 |
| rs12683791 | | 0.001 | 0.936 | 0.030 |
| rs7862653 | | 0.001 | 0.779 | 0.349 |
| rs870535 | | 0.001 | 0.912 | 0.319 |
| rs7944972 | OPCML | 0.001 | 0.852 | 0.002 |
| rs582669 | PKHD1 | 0.001 | 0.927 | 0.637 |
| rs13116006 | | 0.001 | 0.867 | 0.614 |
| rs1424790 | | 0.001 | 0.830 | 0.020 |
| rs1625560 | | 0.001 | 0.898 | 0.614 |
| rs3777102 | NRG2 | 0.001 | 0.777 | 0.857 |
| rs912377 | | 0.001 | 0.807 | 0.891 |
| rs13220430 | EYS | 0.001 | 0.819 | 0.422 |
| rs1363472 | KIAA1024L | 0.001 | 0.836 | <0.001 |
| rs171895 | | 0.001 | 0.934 | 0.041 |
| rs9592493 | PCDH9 | 0.001 | 0.829 | 0.586 |
| rs468471 | RCL1 | 0.001 | 0.350 | 0.554 |
| rs2338871 | LCP2 | 0.001 | 0.741 | 0.143 |
| rs2830957 | | 0.001 | 0.883 | 0.586 |
| rs17718358 | | 0.001 | 0.754 | 0.574 |
| rs9645497 | | 0.001 | 0.815 | 0.688 |
| rs6777976 | OXNAD1 | 0.001 | 0.783 | 0.225 |
| rs38478 | | 0.001 | 0.847 | 0.320 |
| rs763842 | | 0.002 | 0.920 | 0.339 |
| rs12413154 | RHOBTB1 | 0.002 | 0.567 | 0.050 |
| rs6995157 | | 0.002 | 0.574 | 0.388 |
| rs10163354 | ABCC11 | 0.002 | 0.919 | <0.001 |
| rs2504927 | SLC22A3 | 0.002 | 0.890 | 0.523 |
| rs13424541 | ZNF638 | 0.002 | 0.869 | 0.574 |
| rs3176295 | FGF17 | 0.002 | 0.751 | 0.574 |
| rs17479629 | MICAL2 | 0.002 | 0.903 | 0.657 |
| rs9815875 | | 0.002 | 0.752 | 0.349 |
| rs2807304 | TLE4 | 0.002 | 0.801 | 0.422 |
| rs11772485 | | 0.002 | 0.740 | 0.058 |
| rs17098621 | | 0.002 | 0.822 | 0.755 |
| rs2063777 | | 0.002 | 0.797 | 0.672 |
| rs10830089 | | 0.002 | 0.773 | 0.399 |
| rs3766509 | ACP6 | 0.002 | 0.846 | 0.778 |
| rs7267327 | | 0.002 | 0.856 | 0.639 |
| rs17150506 | CSNK1G3 | 0.002 | 0.910 | 0.336 |
| rs2112468 | CSNK1G3 | 0.002 | 0.912 | 0.506 |
| rs4546375 | CSNK1G3 | 0.002 | 0.900 | 0.336 |

SUPPLEMENTARY TABLE 4
Ref-SNP selection Top100 list, (BMC6-BMC0)-(AHC6-AHC0)

| | | ANOVA p- | GenTrain | |
|---|---|---|---|---|
| SNP | Nearest Gene | value | Score | ChiTest100 |
| rs2480 | | <0.001 | 0.912 | 0.511 |
| rs1704405 | EHD4 | <0.001 | 0.757 | 0.586 |
| rs7071851 | PTCHD3 | <0.001 | 0.893 | 0.523 |
| rs204925 | LMO1 | <0.001 | 0.817 | 0.004 |
| rs11916112 | ARHGEF3 | <0.001 | 0.813 | 0.007 |
| rs11041982 | STK33 | <0.001 | 0.882 | 0.058 |
| rs7538377 | PCNXL2 | <0.001 | 0.788 | 0.778 |
| rs1409570 | | <0.001 | 0.695 | 0.185 |
| rs17138899 | ACACA | <0.001 | 0.907 | 0.354 |
| rs2302803 | ACACA | <0.001 | 0.879 | 0.354 |
| rs993743 | | <0.001 | 0.853 | 0.080 |
| rs11187169 | | <0.001 | 0.810 | 0.586 |
| rs4712572 | CDKAL1 | <0.001 | 0.760 | 0.062 |
| rs13176923 | | <0.001 | 0.751 | 0.657 |
| rs13189446 | | <0.001 | 0.824 | 0.688 |
| rs10017447 | | <0.001 | 0.789 | 0.001 |
| rs11101387 | ARHGAP22 | <0.001 | 0.808 | 0.149 |
| rs11213776 | | <0.001 | 0.800 | 0.037 |
| rs1502275 | | <0.001 | 0.727 | 0.424 |
| rs17095168 | | <0.001 | 0.743 | 0.421 |
| rs10833451 | NELL1 | 0.001 | 0.729 | <0.001 |
| rs6047259 | | 0.001 | 0.818 | 0.891 |
| rs807013 | | 0.001 | 0.693 | 0.003 |
| rs9523880 | | 0.001 | 0.943 | 0.079 |
| rs968371 | CSMD1 | 0.001 | 0.811 | 0.670 |
| rs11783921 | | 0.001 | 0.855 | 0.574 |
| rs3104917 | | 0.001 | 0.844 | 0.502 |
| rs3887267 | C9orf3 | 0.001 | 0.868 | 0.688 |
| rs11695576 | | 0.001 | 0.754 | 0.407 |
| rs11193140 | SORCS1 | 0.001 | 0.691 | 0.163 |
| rs3744589 | ACACA | 0.001 | 0.945 | 0.495 |
| rs7729395 | | 0.001 | 0.876 | 0.185 |
| rs7660651 | | 0.001 | 0.929 | 0.203 |
| rs11070879 | MAPK6 | 0.001 | 0.921 | 0.399 |
| rs16843307 | | 0.001 | 0.936 | 0.073 |
| rs758504 | NFIC | 0.001 | 0.795 | 0.502 |
| rs7897931 | | 0.001 | 0.836 | 0.339 |
| rs4810347 | | 0.001 | 0.825 | 0.506 |
| rs11590511 | | 0.001 | 0.817 | 0.001 |
| rs1860904 | | 0.001 | 0.856 | 0.011 |
| rs7677806 | | 0.001 | 0.882 | 0.011 |
| rs2460968 | SAMD12 | 0.001 | 0.805 | 0.079 |
| rs10093536 | | 0.001 | 0.884 | 0.667 |
| rs10803976 | | 0.001 | 0.885 | 0.463 |
| rs10242065 | | 0.001 | 0.830 | 0.018 |
| rs13227663 | | 0.001 | 0.860 | 0.027 |
| rs6748854 | | 0.001 | 0.807 | 0.011 |
| rs12666730 | | 0.001 | 0.897 | 0.857 |
| rs3828760 | FAM46A | 0.001 | 0.885 | 0.004 |
| rs654126 | CSMD1 | 0.001 | 0.909 | 0.108 |
| rs7106565 | | 0.001 | 0.879 | 0.891 |
| rs4579523 | | 0.001 | 0.802 | <0.001 |
| rs10933436 | | 0.001 | 0.770 | 0.339 |
| rs11693862 | | 0.001 | 0.776 | 0.339 |
| rs9558407 | | 0.001 | 0.816 | 0.003 |
| rs10463168 | | 0.001 | 0.911 | 0.659 |
| rs6502774 | TUSC5 | 0.001 | 0.808 | 0.268 |
| rs1546208 | | 0.001 | 0.908 | 0.421 |
| rs3735444 | MAGI2 | 0.001 | 0.806 | 0.285 |
| rs1721073 | | 0.001 | 0.910 | 0.399 |
| rs963080 | | 0.001 | 0.913 | 0.399 |
| rs3811976 | SLCO4C1 | 0.001 | 0.771 | 0.943 |
| rs6891076 | | 0.001 | 0.887 | 0.943 |
| rs10929308 | HEATR7B1 | 0.001 | 0.934 | 0.595 |
| rs353747 | | 0.001 | 0.840 | 0.336 |
| rs12891473 | SRP54 | 0.001 | 0.698 | 0.001 |
| rs6951227 | MAGI2 | 0.001 | 0.849 | 0.088 |
| rs6663310 | | 0.001 | 0.756 | 0.001 |
| rs7127684 | STK33 | 0.001 | 0.897 | 0.024 |
| rs13324043 | | 0.001 | 0.792 | 0.463 |
| rs11638978 | | 0.001 | 0.829 | 0.799 |
| rs10124300 | | 0.001 | 0.784 | 0.042 |
| rs16914660 | ANK3 | 0.001 | 0.842 | 0.667 |
| rs12492974 | | 0.001 | 0.778 | 0.234 |
| rs16838912 | | 0.001 | 0.760 | 0.234 |
| rs13028683 | CDKL4 | 0.001 | 0.793 | 0.424 |
| rs1018966 | CTNND2 | 0.001 | 0.924 | 0.012 |
| rs17318596 | ATP5SL | 0.001 | 0.608 | <0.001 |
| rs4674 | BCKDHA | 0.001 | 0.811 | <0.001 |
| rs3118942 | LPPR1 | 0.001 | 0.838 | 0.027 |
| rs6548940 | | 0.001 | 0.785 | 0.093 |
| rs6753302 | EPAS1 | 0.001 | 0.837 | <0.001 |
| rs236004 | | 0.001 | 0.825 | 0.006 |
| rs2413923 | SHC4 | 0.001 | 0.894 | 0.586 |
| rs10774811 | | 0.001 | 0.847 | 0.094 |
| rs828999 | SLC25A24 | 0.001 | 0.934 | 0.828 |
| rs4787016 | A2BP1 | 0.001 | 0.789 | 0.093 |
| rs17545182 | | 0.001 | 0.770 | 0.039 |
| rs17236914 | | 0.001 | 0.845 | 0.072 |
| rs7243663 | L3MBTL4 | 0.001 | 0.771 | 0.688 |
| rs12735509 | | 0.001 | 0.875 | 0.463 |
| rs12065336 | | 0.001 | 0.728 | 0.290 |
| rs9918378 | | 0.001 | 0.785 | 0.185 |
| rs4236002 | CDKAL1 | 0.001 | 0.931 | 0.336 |
| rs12619647 | SEPT2 | 0.001 | 0.819 | 0.914 |
| rs7313017 | LOC100130825 | 0.001 | 0.512 | 0.021 |
| rs9644620 | LOC100128993 | 0.001 | 0.920 | 0.672 |
| rs2599547 | | 0.002 | 0.902 | 0.137 |
| rs974312 | | 0.002 | 0.748 | 0.495 |
| rs6573513 | PPP2R5E | 0.002 | 0.854 | 0.049 |

SUPPLEMENTARY TABLE 5
Gene-SNP selection Top100 list, AHC6-AHC0

| | Nearest | ANOVA p- | | |
|---|---|---|---|---|
| SNP | Gene | value | GenTrainScore | ChiTest100 |
| rs6802942 | PPP2R3A | <0.001 | 0.891 | 0.495 |
| rs6513775 | PTPRT | <0.001 | 0.776 | 0.088 |
| rs4767020 | RPH3A | <0.001 | 0.743 | 0.495 |
| rs9863749 | C3orf20 | <0.001 | 0.854 | 0.421 |
| rs6035839 | XRN2 | <0.001 | 0.891 | 0.422 |
| rs6082384 | XRN2 | <0.001 | 0.937 | 0.422 |
| rs10982661 | TMOD1 | <0.001 | 0.838 | 0.143 |
| rs10071707 | PDZD2 | <0.001 | 0.919 | 0.587 |
| rs17817463 | DISC1 | <0.001 | 0.733 | 0.421 |
| rs283122 | PDZD2 | <0.001 | 0.866 | 0.099 |
| rs6959021 | PKD1L1 | <0.001 | 0.864 | 0.268 |
| rs9639988 | PKD1L1 | <0.001 | 0.819 | 0.268 |
| rs2345122 | ZKSCAN2 | <0.001 | 0.824 | 0.285 |
| rs13047833 | DSCAM | <0.001 | 0.906 | 0.354 |
| rs4871031 | DEPDC6 | <0.001 | 0.833 | 0.225 |
| rs2371438 | ERBB4 | <0.001 | 0.893 | 0.502 |
| rs940539 | CDC2L6 | <0.001 | 0.823 | 0.185 |
| rs10120342 | PLAA | <0.001 | 0.928 | <0.001 |
| rs2836416 | ERG | 0.001 | 0.830 | 0.857 |
| rs17826507 | PHC3 | 0.001 | 0.791 | 0.080 |
| rs17752921 | TPM1 | 0.001 | 0.850 | 0.291 |
| rs2186716 | ST3GAL4 | 0.001 | 0.760 | 0.574 |
| rs9612266 | BCR | 0.001 | 0.897 | 0.830 |
| rs9559759 | COL4A1 | 0.001 | 0.923 | 0.001 |
| rs11755592 | ZFAND3 | 0.001 | 0.807 | <0.001 |
| rs3128 | CTSH | 0.001 | 0.802 | 0.023 |
| rs2920836 | FRS2 | 0.001 | 0.895 | 0.042 |
| rs4785187 | ZNF423 | 0.001 | 0.841 | 0.871 |
| rs7599195 | OSBPL6 | 0.001 | 0.837 | 0.463 |
| rs7559527 | OSBPL6 | 0.001 | 0.919 | 0.463 |
| rs306410 | ATP8A2 | 0.001 | 0.931 | 0.463 |
| rs408359 | AGPAT1 | 0.001 | 0.887 | 0.162 |
| rs7386 | C11orf48 | 0.001 | 0.523 | 0.075 |
| rs1326270 | PTPRC | 0.001 | 0.808 | 0.339 |
| rs765719 | ALDH6A1 | 0.001 | 0.921 | 0.005 |
| rs2518523 | OR6K6 | 0.001 | 0.765 | <0.001 |
| rs16841047 | OR6K6 | 0.001 | 0.937 | <0.001 |
| rs1124922 | HIP1 | 0.001 | 0.768 | 0.688 |
| rs2071487 | GSTM1 | 0.001 | 0.591 | <0.001 |
| rs2071487 | GSTM1 | 0.001 | 0.591 | <0.001 |
| rs6415084 | LPA | 0.001 | 0.838 | 0.055 |
| rs4146673 | ALK | 0.001 | 0.902 | 0.835 |
| rs8051232 | COQ7 | 0.001 | 0.831 | 0.871 |
| rs11759825 | PACSIN1 | 0.001 | 0.773 | 0.285 |
| rs12991495 | DNMT3A | 0.001 | 0.821 | 0.393 |
| rs6984210 | BMP1 | 0.002 | 0.819 | 0.072 |
| rs634370 | ABI3 | 0.002 | 0.745 | 0.802 |
| rs2236862 | GSTM1 | 0.002 | 0.464 | <0.001 |
| rs2076109 | APOBEC3F | 0.002 | 0.614 | 0.064 |
| rs11637984 | SQRDL | 0.002 | 0.583 | 0.007 |
| rs2302254 | NME1 | 0.002 | 0.769 | 0.234 |
| rs10034673 | GPRIN3 | 0.002 | 0.759 | 0.657 |
| rs16962458 | NECAB2 | 0.002 | 0.786 | 0.943 |
| rs13225749 | PTPRZ1 | 0.002 | 0.845 | 0.502 |
| rs4732874 | ADRA1A | 0.002 | 0.879 | 0.462 |
| rs1631117 | DNAH8 | 0.002 | 0.842 | 0.463 |
| rs17062130 | GPM6A | 0.002 | 0.719 | 0.789 |
| rs7098200 | ADK | 0.002 | 0.931 | 0.195 |
| rs7725698 | MCTP1 | 0.002 | 0.781 | 0.170 |
| rs3743936 | MMP25 | 0.002 | 0.703 | 0.657 |
| rs6141443 | RALY | 0.002 | 0.658 | 0.109 |
| rs7188014 | LITAF | 0.002 | 0.778 | 0.916 |
| rs4558548 | PPP1CB | 0.002 | 0.915 | 0.441 |
| rs3748229 | PIK3AP1 | 0.002 | 0.770 | 0.285 |
| rs989128 | CACNA1G | 0.002 | 0.785 | 0.012 |
| rs8129934 | ADARB1 | 0.002 | 0.835 | 0.495 |
| rs6950693 | PTPRN2 | 0.002 | 0.572 | 0.034 |
| rs7557817 | FHL2 | 0.002 | 0.827 | 0.422 |
| rs10486293 | HDAC9 | 0.002 | 0.941 | 0.036 |
| rs17705427 | DNAJC24 | 0.002 | 0.913 | 0.177 |
| rs296886 | HNRNPK | 0.002 | 0.881 | 0.755 |
| rs822570 | ARHGEF11 | 0.002 | 0.927 | 0.030 |
| rs17294592 | SVIL | 0.002 | 0.729 | 0.574 |
| rs888246 | KIAA0999 | 0.002 | 0.838 | 0.463 |
| rs28528975 | GAL3ST2 | 0.002 | 0.787 | 0.393 |
| rs2240191 | RPH3A | 0.002 | 0.825 | 0.014 |
| rs2236862 | GSTM1 | 0.002 | 0.464 | <0.001 |
| rs2401035 | CCDC59 | 0.002 | 0.887 | 0.755 |
| rs12829066 | ITPR2 | 0.003 | 0.811 | 0.639 |
| rs1560489 | GPRIN3 | 0.003 | 0.902 | 0.268 |
| rs8117456 | KIF16B | 0.003 | 0.882 | 0.657 |
| rs7766388 | WDR27 | 0.003 | 0.849 | 0.424 |
| rs520328 | DSCAML1 | 0.003 | 0.780 | 0.463 |
| rs926561 | AKAP12 | 0.003 | 0.827 | 0.128 |
| rs10516471 | PPP3CA | 0.003 | 0.923 | 0.846 |
| rs13376677 | VAV3 | 0.003 | 0.875 | 0.319 |
| rs882422 | PCSK6 | 0.003 | 0.896 | 0.657 |
| rs7714610 | FSTL4 | 0.003 | 0.727 | 0.143 |
| rs3809449 | FAM177A1 | 0.003 | 0.660 | 0.040 |
| rs2523190 | GNAI1 | 0.003 | 0.804 | 0.234 |
| rs6556312 | RGS14 | 0.003 | 0.706 | 0.586 |
| rs12761450 | ANK3 | 0.003 | 0.890 | 0.778 |
| rs7781413 | TMEM195 | 0.003 | 0.869 | 0.943 |
| rs2532007 | ADCY9 | 0.003 | 0.856 | 0.424 |
| rs814528 | SPTBN4 | 0.003 | 0.778 | 0.079 |
| rs10929587 | ADAM17 | 0.003 | 0.851 | 0.011 |
| rs10495563 | ADAM17 | 0.003 | 0.752 | 0.011 |
| rs2382553 | C9orf93 | 0.003 | 0.891 | 0.285 |
| rs221797 | GIGYF1 | 0.003 | 0.884 | 0.203 |
| rs6963037 | C7orf10 | 0.003 | 0.805 | 0.352 |

SUPPLEMENTARY TABLE 6
Gene-SNP selection Top100 list, BMC6-BMC0

| | Nearest | ANOVA p- | | |
|---|---|---|---|---|
| SNP | Gene | value | GenTrainScore | ChiTest100 |
| rs7591006 | SPAG16 | <0.001 | 0.930 | 0.655 |
| rs151290 | KCNQ1 | <0.001 | 0.762 | 0.463 |
| rs12624282 | C2orf43 | <0.001 | 0.897 | 0.144 |
| rs2102472 | LBH | <0.001 | 0.810 | 0.349 |
| rs6850131 | HSD17B13 | <0.001 | 0.850 | 0.057 |
| rs11735092 | HSD17B13 | <0.001 | 0.842 | 0.164 |
| rs1965869 | FAM13A | <0.001 | 0.916 | 0.441 |
| rs12718455 | SNTG2 | <0.001 | 0.837 | 0.511 |
| rs3132680 | TRIM31 | <0.001 | 0.919 | 0.960 |
| rs9827210 | CNTN4 | <0.001 | 0.880 | 0.888 |
| rs10451237 | RICH2 | <0.001 | 0.736 | 0.587 |
| rs12433712 | SRP54 | <0.001 | 0.922 | 0.891 |
| rs7775864 | SNX14 | <0.001 | 0.882 | 0.425 |
| rs6909767 | SNX14 | <0.001 | 0.942 | 0.425 |
| rs7771612 | SNX14 | <0.001 | 0.927 | 0.425 |
| rs7742691 | SNX14 | <0.001 | 0.862 | 0.425 |
| rs6463016 | PRKAR1B | <0.001 | 0.769 | 0.502 |
| rs12184386 | CUL2 | <0.001 | 0.891 | 0.267 |
| rs17126706 | CPNE8 | <0.001 | 0.840 | 0.234 |
| rs7224186 | ARSG | <0.001 | 0.811 | 0.657 |
| rs3129294 | HLA-DPB2 | <0.001 | 0.803 | 0.141 |
| rs13072512 | FOXP1 | <0.001 | 0.862 | 0.474 |
| rs1883414 | HLA-DPB2 | <0.001 | 0.929 | 0.063 |
| rs6577435 | CAMTA1 | <0.001 | NA | 0.440 |
| rs6713506 | FBXO11 | <0.001 | 0.822 | 0.871 |
| rs9392366 | GMDS | <0.001 | 0.843 | 0.421 |
| rs11219462 | VWA5A | <0.001 | 0.897 | 0.495 |
| rs6454472 | SNX14 | 0.001 | 0.942 | 0.549 |
| rs9444352 | SNX14 | 0.001 | 0.892 | 0.549 |
| rs2858996 | HFE | 0.001 | 0.896 | 0.441 |
| rs707889 | HFE | 0.001 | NA | 0.441 |
| rs989128 | CACNA1G | 0.001 | 0.785 | 0.012 |
| rs1965869 | FAM13A | 0.001 | 0.916 | 0.441 |
| rs7004524 | CSMD1 | 0.001 | 0.849 | 0.018 |
| rs3118860 | DAPK1 | 0.001 | 0.844 | 0.526 |
| rs12034925 | DNAH14 | 0.001 | 0.838 | 0.433 |
| rs7189501 | A2BP1 | 0.001 | 0.843 | 0.421 |
| rs17533945 | MYO9B | 0.001 | 0.817 | 0.799 |
| rs1323080 | C10orf11 | 0.001 | 0.820 | 0.835 |
| rs6775216 | SHOX2 | 0.001 | 0.856 | 0.023 |
| rs31872 | PCDHA11 | 0.001 | 0.822 | 0.937 |
| rs13213129 | LPAL2 | 0.001 | 0.577 | 0.742 |
| rs9282566 | ABCC4 | 0.001 | 0.782 | 0.657 |
| rs169250 | FLJ25076 | 0.001 | 0.792 | 0.441 |
| rs17170270 | TPK1 | 0.001 | 0.913 | 0.495 |
| rs487269 | SRGAP3 | 0.001 | 0.762 | 0.290 |
| rs17170134 | CNTNAP2 | 0.001 | 0.935 | 0.433 |
| rs11598750 | ADARB2 | 0.001 | 0.832 | 0.006 |
| rs370156 | LILRB4 | 0.001 | 0.794 | 0.109 |
| rs6125103 | SULF2 | 0.001 | 0.837 | 0.203 |
| rs4671052 | EHBP1 | 0.001 | 0.842 | 0.291 |
| rs10423215 | ZNF347 | 0.001 | 0.925 | 0.290 |
| rs10814381 | RNF38 | 0.001 | 0.841 | 0.354 |
| rs10824363 | C10orf11 | 0.001 | 0.843 | 0.015 |
| rs6704367 | RP1- | 0.001 | 0.900 | 0.820 |
| | 21O18.1 | | | |
| rs17623914 | PTPRC | 0.001 | 0.921 | 0.001 |
| rs6935269 | C6orf10 | 0.001 | 0.867 | 0.441 |
| rs6594013 | ATP2B4 | 0.001 | 0.838 | 0.285 |
| rs2616693 | CTNNA3 | 0.001 | 0.952 | 0.587 |
| rs2023945 | CCDC46 | 0.001 | 0.765 | 0.742 |
| rs3755930 | CTBP1 | 0.002 | 0.822 | 0.599 |
| rs7577342 | BRE | 0.002 | 0.841 | 0.672 |
| rs1902966 | BRE | 0.002 | 0.878 | 0.672 |
| rs12465000 | BRE | 0.002 | 0.870 | 0.672 |
| rs6594013 | ATP2B4 | 0.002 | 0.838 | 0.285 |
| rs1062470 | CDSN | 0.002 | 0.765 | 0.667 |
| rs1867996 | CDH23 | 0.002 | 0.846 | 0.039 |
| rs16955433 | CMIP | 0.002 | 0.720 | 0.040 |
| rs11667351 | BAX | 0.002 | 0.868 | 0.234 |
| rs1397202 | TAC1 | 0.002 | 0.910 | 0.495 |
| rs17789420 | NPSR1 | 0.002 | 0.769 | 0.943 |
| rs10149561 | FOXN3 | 0.002 | 0.830 | 0.040 |
| rs11574 | ID3 | 0.002 | 0.848 | 0.462 |
| rs2664371 | MMP16 | 0.002 | 0.900 | 0.600 |
| rs1285882 | RREB1 | 0.002 | 0.787 | 0.586 |
| rs2071587 | FOXN1 | 0.002 | 0.730 | 0.742 |
| rs10504965 | PGCP | 0.002 | 0.895 | 0.362 |
| rs17035482 | PEX14 | 0.002 | 0.843 | 0.495 |
| rs11236172 | POLD3 | 0.002 | 0.927 | 0.005 |
| rs17443228 | IMMP2L | 0.002 | 0.699 | 0.657 |
| rs1748356 | PDSS1 | 0.002 | 0.912 | 0.195 |
| rs1780179 | PDSS1 | 0.002 | 0.786 | 0.195 |
| rs1465314 | DTX2 | 0.002 | 0.843 | 0.857 |
| rs13115520 | JAKMIP1 | 0.002 | 0.750 | 0.143 |
| rs7780194 | BBS9 | 0.002 | 0.792 | 0.011 |
| rs8009944 | RAD51L1 | 0.002 | 0.767 | 0.362 |
| rs4843747 | BANP | 0.002 | 0.699 | 0.495 |
| rs6729843 | C2orf43 | 0.002 | 0.911 | 0.185 |
| rs340597 | C2orf43 | 0.002 | 0.756 | 0.143 |
| rs2246618 | MICB | 0.002 | 0.813 | 0.891 |
| rs2269058 | RNF8 | 0.002 | 0.806 | 0.526 |
| rs4235587 | ADCY2 | 0.002 | 0.731 | 0.253 |
| rs9912900 | SLC39A11 | 0.002 | 0.792 | 0.291 |
| rs1796236 | PTPRN2 | 0.002 | 0.685 | 0.040 |
| rs1242787 | PTPRN2 | 0.002 | 0.812 | 0.320 |
| rs353644 | CD44 | 0.002 | 0.870 | 0.267 |
| rs801712 | CERK | 0.002 | 0.807 | 0.586 |
| rs17270501 | RORA | 0.002 | 0.764 | 0.657 |
| rs9507557 | ATP8A2 | 0.002 | 0.919 | 0.177 |
| rs347117 | ADAM10 | 0.003 | 0.895 | 0.548 |

SUPPLEMENTARY TABLE 7
Gene-SNP selection Top100 list, BMC6-AHC6

| | Nearest | ANOVA p- | | |
|---|---|---|---|---|
| SNP | Gene | value | GenTrainScore | ChiTest100 |
| rs12735646 | ARID1A | <0.001 | 0.783 | 0.742 |
| rs12726287 | ARID1A | <0.001 | 0.743 | 0.742 |
| rs10896623 | TIMM10 | <0.001 | 0.924 | 0.058 |
| rs12124339 | CAPZB | <0.001 | 0.752 | 0.352 |
| rs3738919 | ITGAV | <0.001 | 0.925 | 0.595 |
| rs151290 | KCNQ1 | <0.001 | 0.762 | 0.463 |
| rs6802942 | PPP2R3A | <0.001 | 0.891 | 0.495 |
| rs4726075 | PRKAG2 | <0.001 | 0.717 | 0.073 |
| rs11672111 | RDH13 | <0.001 | 0.918 | 0.424 |
| rs12034925 | DNAH14 | <0.001 | 0.838 | 0.433 |
| rs11699888 | SULF2 | <0.001 | 0.862 | 0.421 |
| rs2857107 | HLA-DOB | <0.001 | 0.839 | 0.143 |
| rs2345122 | ZKSCAN2 | <0.001 | 0.824 | 0.285 |
| rs6775216 | SHOX2 | <0.001 | 0.856 | 0.023 |
| rs12913832 | HERC2 | <0.001 | 0.835 | 0.023 |
| rs9863749 | C3orf20 | <0.001 | 0.854 | 0.421 |
| rs9913412 | ALOX15P | <0.001 | 0.668 | 0.168 |
| rs1473114 | NUDCD3 | <0.001 | 0.881 | 0.235 |
| rs1800562 | HFE | <0.001 | 0.787 | 0.004 |
| rs2252551 | C6orf106 | <0.001 | 0.928 | 0.399 |
| rs2814998 | C6orf106 | <0.001 | 0.875 | 0.399 |
| rs13379803 | AKAP13 | <0.001 | 0.912 | 0.295 |
| rs676602 | NALCN | <0.001 | 0.671 | 0.857 |
| rs13182101 | CLTB | <0.001 | 0.900 | 0.295 |
| rs7959125 | ACSS3 | <0.001 | 0.762 | 0.424 |
| rs9289121 | C3orf30 | <0.001 | 0.816 | 0.960 |
| rs10889550 | LEPR | <0.001 | 0.817 | 0.502 |
| rs13213129 | LPAL2 | 0.001 | 0.577 | 0.742 |
| rs341397 | RORA | 0.001 | 0.765 | 0.742 |
| rs6704367 | RP1- | 0.001 | 0.900 | 0.820 |
| | 21O18.1 | | | |
| rs17003153 | FRAS1 | 0.001 | 0.878 | 0.463 |
| rs17170270 | TPK1 | 0.001 | 0.913 | 0.495 |
| rs6780412 | CLDN18 | 0.001 | 0.903 | 0.778 |
| rs4677611 | FOXP1 | 0.001 | 0.846 | 0.137 |
| rs555225 | ANK1 | 0.001 | 0.714 | 0.742 |
| rs16890723 | ANK1 | 0.001 | 0.704 | 0.820 |
| rs2518523 | OR6K6 | 0.001 | 0.765 | 0.000 |
| rs16841047 | OR6K6 | 0.001 | 0.937 | 0.000 |
| rs13061519 | NLGN1 | 0.001 | 0.877 | 0.657 |
| rs2040784 | NFE2L3 | 0.001 | 0.691 | 0.136 |
| rs7521047 | NUP210L | 0.001 | 0.919 | 0.846 |
| rs10807151 | FKBP5 | 0.001 | 0.826 | 0.058 |
| rs8031186 | ADAMTSL3 | 0.001 | 0.874 | 0.109 |
| rs17303530 | RORA | 0.001 | 0.920 | 0.291 |
| rs370156 | LILRB4 | 0.001 | 0.794 | 0.109 |
| rs2482424 | ABCA1 | 0.001 | 0.759 | 0.290 |
| rs10888977 | PPAP2B | 0.001 | 0.837 | 0.399 |
| rs4808571 | MYO9B | 0.001 | 0.731 | 0.225 |
| rs13217929 | SYNJ2 | 0.001 | 0.842 | 0.268 |
| rs17270501 | RORA | 0.001 | 0.764 | 0.657 |
| rs547364 | SLC25A24 | 0.001 | 0.758 | 0.799 |
| rs7103581 | C11orf49 | 0.001 | 0.809 | 0.637 |
| rs7810512 | TBRG4 | 0.001 | 0.874 | 0.141 |
| rs2744957 | C6orf106 | 0.001 | 0.791 | 0.094 |
| rs2814992 | C6orf106 | 0.001 | 0.938 | 0.094 |
| rs7235783 | SPIRE1 | 0.001 | 0.934 | 0.506 |
| rs12516416 | AFF4 | 0.001 | 0.913 | 0.422 |
| rs10988495 | COL15A1 | 0.002 | 0.801 | 0.354 |
| rs17114699 | ANG | 0.002 | 0.820 | 0.143 |
| rs9726956 | FGGY | 0.002 | 0.856 | 0.029 |
| rs760456 | ITGB2 | 0.002 | 0.766 | 0.067 |
| rs2081893 | ZNF541 | 0.002 | 0.773 | 0.820 |
| rs12972658 | ZNF541 | 0.002 | 0.800 | 0.742 |
| rs12361074 | FLJ32810 | 0.002 | 0.811 | 0.502 |
| rs6984210 | BMP1 | 0.002 | 0.819 | 0.072 |
| rs11247287 | PCSK6 | 0.002 | 0.798 | 0.260 |
| rs17718113 | VAT1L | 0.002 | 0.809 | 0.688 |
| rs1418253 | LPHN2 | 0.002 | 0.868 | 0.424 |
| rs367881 | LPHN2 | 0.002 | 0.874 | 0.421 |
| rs4491236 | NTM | 0.002 | 0.854 | 0.899 |
| rs17133676 | OGDH | 0.002 | 0.849 | 0.820 |
| rs2023945 | CCDC46 | 0.002 | 0.765 | 0.742 |
| rs1531817 | PCSK6 | 0.002 | 0.785 | 0.141 |
| rs1573994 | ITPR2 | 0.002 | 0.845 | 0.285 |
| rs3790515 | RORC | 0.002 | 0.775 | 0.495 |
| rs3859534 | LILRA6 | 0.002 | 0.737 | 0.441 |
| rs11259333 | FAM107B | 0.002 | 0.693 | 0.144 |
| rs11967633 | TMEM63B | 0.002 | 0.710 | 0.014 |
| rs2071587 | FOXN1 | 0.002 | 0.730 | 0.742 |
| rs13225749 | PTPRZ1 | 0.002 | 0.845 | 0.502 |
| rs306410 | ATP8A2 | 0.002 | 0.931 | 0.463 |
| rs4775310 | RORA | 0.002 | 0.736 | 0.014 |
| rs320109 | RCOR2 | 0.002 | 0.811 | 0.295 |
| rs155104 | ITGA4 | 0.003 | 0.921 | 0.891 |
| rs11208660 | LEPR | 0.003 | 0.895 | 0.295 |
| rs625014 | RAB31 | 0.003 | 0.818 | 0.164 |
| rs10071707 | PDZD2 | 0.003 | 0.919 | 0.587 |
| rs222857 | CLDN7 | 0.003 | 0.807 | 0.234 |
| rs12582168 | NCOR2 | 0.003 | 0.817 | 0.051 |
| rs112544 | LZTR1 | 0.003 | 0.870 | 0.128 |
| rs1415701 | L3MBTL3 | 0.003 | 0.862 | 0.724 |
| rs995435 | TGFBR2 | 0.003 | 0.843 | 0.393 |
| rs7770046 | TMEM181 | 0.003 | 0.781 | 0.185 |
| rs3751909 | FOXK2 | 0.003 | 0.731 | 0.352 |
| rs17128050 | GCH1 | 0.003 | 0.899 | 0.463 |
| rs10423215 | ZNF347 | 0.003 | 0.925 | 0.290 |
| rs2186716 | ST3GAL4 | 0.003 | 0.760 | 0.574 |
| rs10989419 | RP11- | 0.003 | 0.859 | 0.058 |
| | 35N6.1 | | | |
| rs7781464 | CNTNAP2 | 0.003 | 0.876 | 0.135 |
| rs2238202 | RGS6 | 0.003 | 0.925 | 0.295 |

SUPPLEMENTARY TABLE 8
Gene-SNP selection Top100 list, (BMC6-BMC0)-(AHC6-AHC0)

| | Nearest | ANOVA p- | | |
|---|---|---|---|---|
| SNP | Gene | value | GenTrainScore | ChiTest100 |
| rs7210402 | SGSM2 | <0.001 | 0.839 | 0.863 |
| rs1806516 | P2RY6 | <0.001 | 0.694 | 0.914 |
| rs2345122 | ZKSCAN2 | <0.001 | 0.824 | 0.285 |
| rs7004524 | CSMD1 | <0.001 | 0.849 | 0.018 |
| rs17817463 | DISC1 | <0.001 | 0.733 | 0.421 |
| rs11623922 | KCNK13 | <0.001 | 0.751 | 0.064 |
| rs370133 | NRCAM | <0.001 | 0.880 | 0.511 |
| rs341397 | RORA | <0.001 | 0.765 | 0.742 |
| rs2427638 | PCMTD2 | <0.001 | 0.824 | 0.064 |
| rs7224186 | ARSG | <0.001 | 0.811 | 0.657 |
| rs11672111 | RDH13 | <0.001 | 0.918 | 0.424 |
| rs10889550 | LEPR | <0.001 | 0.817 | 0.502 |
| rs7203078 | CMIP | <0.001 | 0.753 | 0.574 |
| rs12451892 | SGSM2 | <0.001 | 0.826 | 0.141 |
| rs7203568 | WWOX | <0.001 | 0.815 | 0.778 |
| rs4511641 | RTN2 | 0.001 | 0.799 | 0.339 |
| rs13379803 | AKAP13 | 0.001 | 0.912 | 0.295 |
| rs10888977 | PPAP2B | 0.001 | 0.837 | 0.399 |
| rs2343869 | SSPN | 0.001 | 0.845 | 0.318 |
| rs845204 | CAMTA1 | 0.001 | 0.930 | 0.063 |
| rs11079323 | MSI2 | 0.001 | 0.928 | 0.009 |
| rs3738919 | ITGAV | 0.001 | 0.925 | 0.595 |
| rs12460755 | INSR | 0.001 | 0.927 | 0.433 |
| rs7235783 | SPIRE1 | 0.001 | 0.934 | 0.506 |
| rs10071707 | PDZD2 | 0.001 | 0.919 | 0.587 |
| rs1018788 | LARGE | 0.001 | 0.895 | 0.474 |
| rs4335165 | MTUS1 | 0.001 | 0.753 | 0.287 |
| rs389883 | STK19 | 0.001 | 0.902 | 0.522 |
| rs4801163 | ZNF667 | 0.001 | 0.936 | 0.177 |
| rs9304776 | ZNF667 | 0.001 | 0.852 | 0.177 |
| rs13225749 | PTPRZ1 | 0.001 | 0.845 | 0.502 |
| rs4384073 | DDX58 | 0.001 | 0.778 | 0.203 |
| rs2836416 | ERG | 0.001 | 0.830 | 0.857 |
| rs9346818 | LPAL2 | 0.001 | 0.891 | 0.522 |
| rs3804267 | PPAP2A | 0.001 | 0.918 | 0.830 |
| rs12246732 | FAM107B | 0.001 | 0.869 | 0.225 |
| rs6785790 | SETD2 | 0.001 | 0.854 | 0.048 |
| rs16869706 | SLIT2 | 0.001 | 0.897 | 0.655 |
| rs10989419 | RP11- | 0.001 | 0.859 | 0.058 |
| | 35N6.1 | | | |
| rs9853081 | FOXP1 | 0.001 | 0.914 | 0.001 |
| rs7712431 | CSNK1A1 | 0.001 | 0.932 | 0.655 |
| rs6415084 | LPA | 0.002 | 0.838 | 0.055 |
| rs7076232 | BTBD16 | 0.002 | 0.805 | 0.587 |
| rs11814901 | BTBD16 | 0.002 | 0.835 | 0.587 |
| rs2481665 | INADL | 0.002 | 0.815 | 0.003 |
| rs7625067 | SETD2 | 0.002 | 0.813 | 0.009 |
| rs2071587 | FOXN1 | 0.002 | 0.730 | 0.742 |
| rs10426628 | SULT2B1 | 0.002 | 0.677 | 0.871 |
| rs3893677 | KCTD1 | 0.002 | 0.809 | 0.891 |
| rs2010010 | GALNT10 | 0.002 | 0.777 | 0.090 |
| rs2176771 | MMP16 | 0.002 | 0.675 | 0.080 |
| rs12034925 | DNAH14 | 0.002 | 0.838 | 0.433 |
| rs17170270 | TPK1 | 0.002 | 0.913 | 0.495 |
| rs9390569 | SASH1 | 0.002 | 0.930 | 0.506 |
| rs11208660 | LEPR | 0.002 | 0.895 | 0.295 |
| rs164577 | SLC30A5 | 0.002 | 0.840 | 0.001 |
| rs169250 | FLJ25076 | 0.002 | 0.792 | 0.441 |
| rs2260000 | BAT2 | 0.002 | 0.810 | 0.526 |
| rs2736172 | BAT2 | 0.002 | 0.728 | 0.526 |
| rs10814381 | RNF38 | 0.002 | 0.841 | 0.354 |
| rs1133195 | MXI1 | 0.002 | 0.905 | 0.291 |
| rs2298229 | OLFM4 | 0.002 | 0.909 | 0.399 |
| rs10979586 | IKBKAP | 0.002 | 0.935 | 0.260 |
| rs1883414 | HLA-DPB2 | 0.002 | 0.929 | 0.063 |
| rs2371438 | ERBB4 | 0.002 | 0.893 | 0.502 |
| rs2010576 | MICAL2 | 0.002 | 0.819 | 0.549 |
| rs550338 | SOX5 | 0.002 | 0.879 | 0.043 |
| rs788332 | MYH14 | 0.002 | 0.789 | 0.138 |
| rs9726956 | FGGY | 0.002 | 0.856 | 0.029 |
| rs8087174 | OSBPL1A | 0.002 | 0.869 | 0.639 |
| rs151290 | KCNQ1 | 0.002 | 0.762 | 0.463 |
| rs3094476 | KCTD5 | 0.002 | 0.850 | 0.001 |
| rs876687 | TGFBR2 | 0.002 | 0.847 | 0.502 |
| rs3773661 | TGFBR2 | 0.002 | 0.794 | 0.495 |
| rs6775216 | SHOX2 | 0.003 | 0.856 | 0.023 |
| rs7901290 | CAMK1D | 0.003 | 0.900 | 0.672 |
| rs3809572 | SMAD3 | 0.003 | 0.726 | 0.495 |
| rs2186716 | ST3GAL4 | 0.003 | 0.760 | 0.574 |
| rs11967633 | TMEM63B | 0.003 | 0.710 | 0.014 |
| rs6925303 | FYN | 0.003 | 0.882 | 0.019 |
| rs6914091 | FYN | 0.003 | 0.713 | 0.019 |
| rs6930230 | FYN | 0.003 | 0.933 | 0.019 |
| rs555225 | ANK1 | 0.003 | 0.714 | 0.742 |
| rs16890723 | ANK1 | 0.003 | 0.704 | 0.820 |
| rs11853311 | SLCO3A1 | 0.003 | 0.799 | 0.440 |
| rs6650615 | MPPE1 | 0.003 | 0.916 | 0.290 |
| rs1133195 | MXI1 | 0.003 | 0.905 | 0.291 |
| rs2286294 | GLI3 | 0.003 | 0.959 | 0.137 |
| rs17799872 | ADCY3 | 0.003 | 0.746 | 0.421 |
| rs2744805 | RIMS3 | 0.003 | 0.785 | 0.614 |
| rs3016562 | PARK2 | 0.003 | 0.852 | 0.009 |
| rs6868292 | PPAP2A | 0.003 | 0.876 | 0.290 |
| rs16924332 | ABCC9 | 0.003 | 0.937 | 0.778 |
| rs2201945 | PCDH7 | 0.003 | 0.844 | 0.295 |
| rs10010739 | PCDH7 | 0.003 | 0.899 | 0.030 |
| rs2285431 | HDAC9 | 0.003 | 0.837 | 0.360 |
| rs10503284 | CSMD1 | 0.003 | 0.719 | 0.143 |
| rs3774491 | CACNA1D | 0.003 | 0.839 | 0.888 |
| rs2518523 | OR6K6 | 0.003 | 0.765 | <0.001 |
| rs16841047 | OR6K6 | 0.003 | 0.937 | <0.001 |

1. A method of screening individuals at risk of developing insulin resistance comprising analyzing chromosomal DNA taken from the individual for the presence of at least one single nucleotide polymorphism (SNP) selected from the group consisting of
```
rs16961756:
(SEQ ID NO: 1)
cgggccttcctcgccagcacctccattcct[a/g]aggctcacgtgggag
agacagtgtggagag,
    Chr17:17359619, G→A;
rs1242483:
(SEQ ID NO: 2)
GAAGTTAAGAGTAAATAAAATAGTCA[C/T]GTTTGGGAGCATGAAGGAG
CCGGCA,
    Chr17:17351675, T→C;
rs29095:
(SEQ ID NO: 3)
tccctcgaataaaggtgaaatttttaaaat[a/c]tcagtgaataggaat
gtgcaaagctctaag,
    Chr18:9957549, C→A;
rs7237794:
(SEQ ID NO: 4)
ctgcccctcccgacacagcacatacacaca[c/t]tgacgttttgctact
acagcatatagcctt,
    Chr18:9951304, T→C;
rs917688:
(SEQ ID NO: 5)
ttctctcatgcttaatatttggaactataa[a/c]gctaaaggccattga
cgtagctaaaaatct,
    Chr18:9962736, C→A;
rs6494711:
(SEQ ID NO: 7)
aaaattgaggaaaatcccagaagatagagc[c/t]aaaagacaagagatg
taaaaatgcacaagg,
    Chr15:68374027, T→C; and
rs1489595:
(SEQ ID NO: 8)
AATTTTCTGTTTACACAAGTGATTCT[A/G]TAAGCAAACCAGGGTTCTC
CATGGT,
    (Chr15:68377126, A→G);
```
wherein an individual heterozygous or homozygous for at least one SNP is identified as having an increased risk of developing insulin resistance.

2. A method of screening individuals at risk of developing type II diabetes (T2D) comprising analyzing chromosomal DNA taken from the individual for the presence of at least one single nucleotide polymorphism (SNP) selected from the group consisting of
```
rs16961756:
(SEQ ID NO: 1)
cgggccttcctcgccagcacctccattcct[a/g]aggctcacgtgggag
agacagtgtggagag,
    Chr17:17359619, G→A;
rs1242483:
(SEQ ID NO: 2)
GAAGTTAAGAGTAAATAAAATAGTCA[C/T]GTTTGGGAGCATGAAGGAG
CCGGCA,
    Ch+r17:17351675, T→C;
rs29095:
(SEQ ID NO: 3)
tccctcgaataaaggtgaaatttttaaaat[a/c]tcagtgaataggaat
gtgcaaagctctaag,
    Chr18:9957549, C→A;
rs7237794:
(SEQ ID NO: 4)
ctgcccctcccgacacagcacatacacaca[c/t]tgacgttttgctact
acagcatatagcctt,
    Chr18:9951304, T→C;
rs917688:
(SEQ ID NO: 5)
ttctctcatgcttaatatttggaactataa[a/c]gctaaaggccattga
cgtagctaaaaatct,
    Chr18:9962736, C→A;
rs6494711:
(SEQ ID NO: 7)
aaaattgaggaaaatcccagaagatagagc[c/t]aaaagacaagagatg
taaaaatgcacaagg,
    Chr15:68374027, T→C; and
rs1489595:
(SEQ ID NO: 8)
AATTTTCTGTTTACACAAGTGATTCT[A/G]TAAGCAAACCAGGGTTCTC
CATGGT,
    (Chr15:68377126, A→G);
```
wherein an individual heterozygous or homozygous for at least one SNP is identified as having an increased risk of developing T2D.

3. A method of identifying single nucleotide polymorphisms (SNPs) associated with insulin resistance comprising identifying SNPs linked with vesicle-associated membrane protein-associated protein A (VAPA) or protein inhibitor of activated STAT-1 (PIAS1) that are present in individuals having insulin resistant cells at statistically significant levels compared to individuals without insulin resistant cells.

4. A method of screening individuals at risk of developing insulin resistance comprising analyzing chromosomal DNA taken from the individual for the presence of a single nucleotide polymorphism (SNP) rs2291726: TCTAGGGACACTTGAATCTTTTAATA[C/T]CTGAACCCCAAAAGCAGAGGGTACC, (SEQ ID NO: 9) Chr17:47039254, C→T,
wherein an individual heterozygous or homozygous for the SNP is identified as having an increased risk of developing insulin resistance.

5. A method of screening individuals at risk of developing type II diabetes (T2D) comprising analyzing chromosomal DNA taken from the individual for the presence of a single nucleotide polymorphism (SNP) rs2291726: TCTAGGGACACTTGAATCTTTTAATA[C/T]CTGAACCCCAAAAGCAGAGGGTACC, (SEQ ID NO: 9) Chr17:47039254, C→T,
wherein an individual heterozygous or homozygous for the SNP is identified as having an increased risk of developing T2D.

6. A method of identifying single nucleotide polymorphisms (SNPs) associated with insulin resistance comprising:
providing a test population of healthy individuals with a body mass index of between 24.5 and 27.5 that undergo two interventions, wherein the first intervention is feeding on a high-carbohydrate diet and the second intervention is feeding on a moderate-carbohydrate diet for two test periods separated with ordinary eating habits;
collecting fasting blood samples from individuals before and after each test period;
analyzing the fasting blood samples for leukocyte gene expression levels and insulin resistance, wherein plasma protein levels are analyzed for visfatin, resistin, insulin, C-peptide, glucagon, plasminogen activator inhibitor-1, glucagon-like peptide-1, tumor necrosis factor alpha, interleukin-6, ghrelin, leptin, and gastric inhibitory polypeptide (GIP);
performing pairwise comparisons (a) between results of the analysis of the individuals of the first intervention after and before the test period; (b) between results of the analysis of the individuals of the second intervention after and before the test period; (c) between results of the analysis of the individuals of the first intervention after the test period and results of the analysis of the individuals of the second intervention after the test period; and (d) between (a) and (b); and
identifying differentially expressed genes in response to each diet intervention period;
genotyping all individuals in loci linked to the differentially expressed genes; and
performing a statistical analysis to determine SNPs significantly correlated with insulin resistance in individuals of the test population.

7. A method for screening for candidate genes for molecular mechanisms involved in insulin resistance comprising the use of VAPA and plasma protein inhibitor of activated STAT-1 (PIAS 1).

8. A method for diagnosing insulin resistance correlated a dietary disease comprising testing an individual's genomic DNA for the presence of at least one single nucleotide polymorphism (SNP) selected from the group consisting of
```
rs16961756:
(SEQ ID NO: 1)
cgggccttcctcgccagcacctccattcct[a/g]aggctcacgtgggag
agacagtgtggagag,
    Chr17:17359619, G→A;
rs1242483:
(SEQ ID NO: 2)
GAAGTTAAGAGTAAATAAAATAGTCA[C/T]GTTTGGGAGCATGAAGGAG
CCGGCA,
    Chr17:17351675, T→C;
rs29095:
(SEQ ID NO: 3)
tccctcgaataaaggtgaaatttttaaaat[a/c]tcagtgaataggaat
gtgcaaagctctaag,
    Chr18:9957549, C→A;
rs7237794:
(SEQ ID NO: 4)
ctgcccctcccgacacagcacatacacaca[c/t]tgacgttttgctact
acagcatatagcctt,
    Chr18:9951304, T→C;
rs917688:
(SEQ ID NO: 5)
ttctctcatgcttaatatttggaactataa[a/c]gctaaaggccattga
cgtagctaaaaatct,
    Chr18:9962736, C→A;
rs6494711:
(SEQ ID NO: 7)
aaaattgaggaaaatcccagaagatagagc[c/t]aaaagacaagagatg
taaaaatgcacaagg
    Chr15:68374027, T→C and
rs1489595:
(SEQ ID NO: 8)
AATTTTCTGTTTACACAAGTGATTCT[A/G]TAAGCAAACCAGGGTTCTC
CATGGT,
    (Chr15:68377126, A→G).
```

9. The method according to claim 8, wherein the dietary disease is associated with glycemic load.

10. A method of developing drugs for regulating an individual's glycemic response comprising using a marker selected from the group consisting of vesicle-associated membrane protein-associated protein A (VAPA), plasma protein inhibitor of activated STAT-1 (PIAS1),
```
rs16961756:
(SEQ ID NO: 1)
cgggccttcctcgccagcacctccattcct[a/g]aggctcacgtgggag
agacagtgtggagag,
    Chr17:17359619, G→A;
rs1242483:
(SEQ ID NO: 2)
GAAGTTAAGAGTAAATAAAATAGTCA[C/T]GTTTGGGAGCATGAAGGAG
CCGGCA,
    Chr17:17351675, T→C;
rs29095:
(SEQ ID NO: 3)
tccctcgaataaaggtgaaatttttaaaat[a/c]tcagtgaataggaat
gtgcaaagctctaag,
    Chr18:9957549, C→A;
rs7237794:
(SEQ ID NO: 4)
ctgcccctcccgacacagcacatacacaca[c/t]tgacgttttgctact
acagcatatagcctt,
    Chr18:9951304, T→C;
rs917688:
(SEQ ID NO: 5)
ttctctcatgcttaatatttggaactataa[a/c]gctaaaggccattga
cgtagctaaaaatct,
    Chr18:9962736, C→A;
rs6494711:
(SEQ ID NO: 7)
aaaattgaggaaaatcccagaagatagagc[c/t]aaaagacaagagatg
taaaaatgcacaagg
    Chr15:68374027, T→C and
rs1489595:
(SEQ ID NO: 8)
AATTTTCTGTTTACACAAGTGATTCT[A/G]TAAGCAAACCAGGGTTCTC
CATGGT,
    (Chr15:68377126, A→G).
```

11. A method for providing a dietary plan for an individual genetically predisposed to type II diabetes (T2D) comprising,
performing genomic typing of the individual's genomic DNA, wherein the typing comprises testing the individual's genomic DNA for the presence of at least one single nucleotide polymorphism (SNP) selected from the group consisting of
```
rs16961756:
(SEQ ID NO: 1)
cgggccttcctcgccagcacctccattcct[a/g]aggctcacgtgggag
agacagtgtggagag,
    Chr17:17359619, G→A;
rs1242483:
(SEQ ID NO: 2)
GAAGTTAAGAGTAAATAAAATAGTCA[C/T]GTTTGGGAGCATGAAGGAG
CCGGCA,
    Chr17:17351675, T→C;
rs29095:
(SEQ ID NO: 3)
tccctcgaataaaggtgaaatttttaaaat[a/c]tcagtgaataggaat
gtgcaaagctctaag,
    Chr18:9957549, C→A;
rs7237794:
(SEQ ID NO: 4)
ctgcccctcccgacacagcacatacacaca[c/t]tgacgttttgctact
acagcatatagcctt,
    Chr18:9951304, T→C;
rs917688:
(SEQ ID NO: 5)
ttctctcatgcttaatatttggaactataa[a/c]gctaaaggccattga
cgtagctaaaaatct,
    Chr18:9962736, C→A;
rs6494711:
(SEQ ID NO: 7)
aaaattgaggaaaatcccagaagatagagc[c/t]aaaagacaagagatg
taaaaatgcacaagg
    Chr15:68374027, T→C; and
rs1489595:
(SEQ ID NO: 8)
AATTTTCTGTTTACACAAGTGATTCT[A/G]TAAGCAAACCAGGGTTCTC
CATGGT,
    (Chr15:68377126, A→G); and
```
providing the dietary plan based on the individual's genomic type.

12. A method for analyzing an individual's physiological response to dietary glycemic load comprising,
performing genomic typing of the individual's genomic DNA, wherein the typing comprises testing the individual's genomic DNA for the presence of at least one single nucleotide polymorphism (SNP) selected from the group consisting of
```
rs16961756:
(SEQ ID NO: 1)
cgggccttcctcgccagcacctccattcct[a/g]aggctcacgtgggag
agacagtgtggagag,
    Chr17:17359619, G→A;
rs1242483:
(SEQ ID NO: 2)
GAAGTTAAGAGTAAATAAAATAGTCA[C/T]GTTTGGGAGCATGAAGGAG
CCGGCA,
    Chr17:17351675, T→C;
rs29095:
(SEQ ID NO: 3)
tccctcgaataaaggtgaaatttttaaaat[a/c]tcagtgaataggaat
gtgcaaagctctaag,
    Chr18:9957549, C→A;
rs7237794:
(SEQ ID NO: 4)
ctgcccctcccgacacagcacatacacaca[c/t]tgacgttttgctact
acagcatatagcctt,
    Chr18:9951304, T→C;
rs917688:
(SEQ ID NO: 5)
ttctctcatgcttaatatttggaactataa[a/c]gctaaaggccattga
cgtagctaaaaatct,
    Chr18:9962736, C→A;
rs6494711:
(SEQ ID NO: 7)
aaaattgaggaaaatcccagaagatagagc[c/t]aaaagacaagagatg
taaaaatgcacaagg
    Chr15:68374027, T→C and
rs1489595:
(SEQ ID NO: 8)
AATTTTCTGTTTACACAAGTGATTCT[A/G]TAAGCAAACCAGGGTTCTC
CATGGT,
    (Chr15:68377126, A→G).
```

13. Use of vesicle-associated membrane protein-associated protein A (VAPA) and plasma protein inhibitor of activated STAT-1 (PIAS1) as candidate genes for molecular mechanisms involved in insulin resistance.

14. Use of the genetic identified genetic SNP markers according to this invention in the diagnosis of insulin resistance correlated with dietary diseases, especially glycemic loads.

15. Use of such markers according to claim 13 developing suitable drugs for regulating glycemic response in people with such diseases.

16. Use of such markers according to claim 13 to explain individual physiological responses to dietary glycemic load characterized by such single nucleotide polymorphism (SNP) typing to provide concrete dietary advice to persons genetically predisposed to type II diabetes (T2D).

17. Use of such markers according to claim 14 developing suitable drugs for regulating glycemic response in people with such diseases.

18. Use of such markers according to claim 14 to explain individual physiological responses to dietary glycemic load characterized by such single nucleotide polymorphism (SNP) typing to provide concrete dietary advice to persons genetically predisposed to type II diabetes (T2D).


SEQUENCE LISTING (9 sequences)

SEQ ID NO: 1 - DNA, 61 bp, Homo sapiens
  misc_feature: rs16961756
cgggccttcctcgccagcacctccattcctraggctcacgtgggagagacagtgtggagag

SEQ ID NO: 2 - DNA, 52 bp, Homo sapiens
  misc_feature: rs1242483
gaagttaagagtaaataaaatagtcaygtttgggagcatgaaggagccggca

SEQ ID NO: 3 - DNA, 61 bp, Homo sapiens
  misc_feature: rs29095
tccctcgaataaaggtgaaatttttaaaatmtcagtgaataggaatgtgcaaagctctaag

SEQ ID NO: 4 - DNA, 61 bp, Homo sapiens
  misc_feature: rs7237794
ctgcccctcccgacacagcacatacacacaytgacgttttgctactacagcatatagcctt

SEQ ID NO: 5 - DNA, 61 bp, Homo sapiens
  misc_feature: rs917688
ttctctcatgcttaatatttggaactataamgctaaaggccattgacgtagctaaaaatct

SEQ ID NO: 6 - DNA, 52 bp, Homo sapiens
  misc_feature: rs10803976
cattaaaagctatcatctaacattgcytggagtgtttatttttaagtgcata

SEQ ID NO: 7 - DNA, 61 bp, Homo sapiens
  misc_feature: rs6494711
aaaattgaggaaaatcccagaagatagagcyaaaagacaagagatgtaaaaatgcacaagg

SEQ ID NO: 8 - DNA, 52 bp, Homo sapiens
  misc_feature: rs1489595
aattttctgtttacacaagtgattctrtaagcaaaccagggttctccatggt

SEQ ID NO: 9 - DNA, 52 bp, Homo sapiens
  misc_feature: rs2291726
tctagggacacttgaatcttttaatayctgaaccccaaaagcagagggtacc